(19) United States
(12) Patent Application Publication
(10) Pub. No.: US 2023/0235310 A1
(43) Pub. Date: Jul. 27, 2023

(54) SYNTHETIC SANTALENE SYNTHASES

(71) Applicant: ISOBIONICS B.V., Geleen (NL)

(72) Inventors: Matthew Quinn STYLES, Geleen (NL); Susan Eline BOUWMEESTER, Geleen (ML); Niels WILLEMS, Geleen (NL); Elena MELILLO, Geleen (NL)

(21) Appl. No.: 18/007,813

(22) PCT Filed: Jun. 1, 2021
(86) PCT No.: PCT/EP2021/064642

§ 371 Date: Dec. 2, 2022

(30) Foreign Application Priority Data
Jun. 4, 2020 (EP) 20178333.9
Mar. 2, 2021 (EP) 21160103.4

(51) Int. Cl. C12N 9/88; C12P 5/00; C12P 7/02

(52) U.S. Cl. CPC C12N 9/88; C12P 5/007; C12P 7/02; C12Y 402/03083

(57) ABSTRACT

Disclosed are santalene synthases with improved product profile and methods for improving santalene synthases. The invention further relates to santalene compositions produced by fermentation that have a greater beta-santalene content than alpha-santalene content.

Santalene synthases are terpene synthases that catalyse the conversion of farnesyl diphosphate (FPP) to a wide range of compounds, including santalenes, for example α-santalene, santalene and epi-β-santalene.

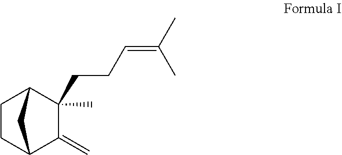

Formula I is a representation of (−)-β-santalene (CAS number 511-59-1; hereinafter referred to as beta-santalene)

Santalene synthases start with the substrate farnesyl pyrophosphate but typically produce a mixture of sesquiterpene products. Typically, a santalene synthase will produce (−)-α-santalene (CAS number 512-61-8; herein after referred to as alpha-santalene) as a main product, followed by either beta-santalene (see formula I) and/or trans-α-bergamotene (CAS number 13474-59-4; herein after also referred to as bergamotene) as the second and third most abundant product. The amounts produced depend on the particular enzyme, and also if beta-santalene is the second most abundant one or bergamotene, but alpha-santalene is dominant in the oils available so far.

Several genes encoding for santalene synthase have been reported (see for example international patent applications WO2018/160066 and references therein). Moreover, these santalene synthase produces a spectrum of santalene sesquiterpenes (comprising most notably beta-santalene, alpha-santalene, epi-β-santalene, bergamotene and beta-bisabolene).

Santalene synthases producing alpha-santalene as the main product are known, e.g. from WO201100026 and Jones et al. (2011) (“Sandalwood fragrance biosynthesis involves sesquiterpene synthases of both the terpene synthase (TPS)-a and TPS-b subfamilies, including santalene synthases.” Jones C. G., Moniodis J., Zulak K. G., et al., The Journal of biological chemistry volume 286 issue 20 pages 17445-17454 May 20, 2011; DOI: 10.1074/jbc.M111.231787) describe terpene synthases from three different Santalum species (Santalum album, S. austrocaledonicum, and S. spicatum) producing α-santalene, α-trans-bergamotene, epi-β-santalene and β-santalene concurrently. The international patent application WO201100026 disclosed in FIG. 1 and table 2 data that is open to the possible misinterpretation that more beta-santalene than alpha-santalene would have been present, e.g. by the showing data from non-quantitative GC-MS analysis without pointing out the unreliability for quantification of this method with respect to amounts of the compounds. Within the same disclosure it is clearly demonstrated to the person skilled in the art that the reverse (more alpha-santalene than beta-santalene) was the case—as is shown in table 2, column 4 of WO201100026 showing the reliable quantitative GC-FID data and table 1 of WO201100026 reporting a more than two-times surplus of alpha-santalene over beta-santalene in natural sandalwood oil.

A surplus of alpha-santalene rather than beta-santalene has also been reported for the same enzymes in the following publication of the researchers behind WO201100026, published also in 2011 and hence presumably with the same data basis: “Sandalwood fragrance biosynthesis involves sesquiterpene synthases of both the terpene synthase (TPS)-a and TPS-b subfamilies, including santalene synthases.” Jones C. G., Moniodis J., Zulak K. G., et al., The Journal of biological chemistry, volume 286 issue 20 pages 17445-17454 May 20, 2011; DOI: 10.1074/jbc.M111.231787. The supplementary material of this article, as well as the corrections of figures of the initial publication (see: Erratum: Sandalwood fragrance biosynthesis involves sesquiterpene synthases of both the terpene synthase (TPS)-a and TPS-b subfamilies, including santalene synthases (Journal of Biological Chemistry (2011) 286 (17445-17454)), Journal of Biological Chemistry volume 287 issue 45 pages 37713-37714 2012, DOI: 10.1074/jbc.A111.231787s) corroborate the fact that more alpha-santalene than beta-santalene was observed by these researchers. Subsequent publications by these researchers confirmed that natural sandalwood oil has no excess of beta-santalene over alpha-santalene (Moniodis et al. 2017 “Sesquiterpene Variation in West Australian Sandalwood (Santalum spicatum)”; Molecules 2017; 22(6)). The known santalene synthases produce more alpha-santalene than beta-santalene (Diaz-Chavez et al. 2013, “Biosynthesis of Sandalwood Oil: Santalum album CYP76F Cytochromes P450 Produce Santalols and Bergamotol”, PLoS ONE, 2013; 8(9)), even when heterologously expressed in tobacco plants (Yin J L, Wong W S (2019) “Production of santalenes and bergamotene in Nicotiana tabacum plants.” PLOS ONE 14(1): e0203249. https://doi.org/10.1371/journal.pone.0203249).

The international patent application published as WO2015153501 describes modified santalene synthase enzymes derived from the S. album santalene synthase with increased terpene synthase activity when compared to the native S. album santalene synthase, yet still an excess of alpha-santalene over beta-santalene, and a santalene synthases with high product profile for alpha-santalene has been discovered (Schalk, M., 2011. Method for Producing Alpha-Santalene. US Pat 2011/008836 A1; international patent application published as WO2018160066). The international patent application published as WO2010/067309 describes a method for producing β-santalene using a santalene synthase from Santalum (Schalk, 2014). U.S. Pat. No. 8,993,284, but still with alpha santalene in excess of beta-santalene.

Hence, with known enzymes beta-santalene is produced always in smaller amounts compared to alpha-santalene, and there are no known examples of a santalene synthase with greater product profile for beta-santalene than alpha-santalene in vivo.

The products of a santalene synthase can be oxidized biosynthetically or chemically to yield their respective santalene alcohols; alpha-santalol, beta-santalol and epi-beta-santalol. Santalols are the main components of sandalwood oil, a highly valued naturally occurring fragrance, which is an important ingredient in perfumes, cosmetics, toiletries, aromatherapy and pharmaceuticals. It has a soft, sweet-woody and balsamic odour that is predominantly imparted from the sesquiterpene alcohols alpha-santalol and beta-santalol. In particular, beta-santalol is regarded as imparting the most important olfactory note of sandalwood. A synthase with greater specificity for beta-santalene is desirable because the product could be oxidized into an oil with high beta-santalol content.

The currently known santalene synthases have a number of distinct drawbacks which are in particular undesirable when they are applied in an industrial santalene production process wherein santalene (and possibly subsequently santalol and in particular β-santalol) is prepared from FPP, either in an isolated reaction (in vitro), e.g. using an isolated santalene synthase or (permeabilized) whole cells, or otherwise, e.g. in a fermentative process being part of a longer metabolic pathway eventually leading to the production of β-santalene from sugar (in vivo).

It may also be advantageous for some applications if the enzyme would produce less alpha-santalene and more trans-α-bergamotene.

Being able to steer the product ratios of the three major products of santalene synthases according to a particular need is desirable.

The invention discloses that surprisingly by relatively simple changes the flexibility of a certain part of the tertiary structure of santalene synthases the product profile of the santalene synthase can be improved. Some of these improved santalene synthases produce beta-santalene and sometimes bergamotene in excess of alpha-santalene, others have increased alpha-santalene production compared to the wildtype enzyme, and they are useful in the production of these compounds for example in large scale industrial processes.

### DETAILED DESCRIPTION OF THE INVENTION

The terms “essentially”, “about”, “approximately”, “substantially” and the like in connection with an attribute or a value, particularly also define exactly the attribute or exactly the value, respectively. The term “substantially” in the context of the same functional activity or substantially the same function means a difference in function preferably within a range of 20%, more preferably within a range of 10%, most preferably within a range of 5% or less compared to the reference function. In context of formulations or compositions, the term “substantially” (e.g., “composition substantially consisting of compound X”) may be used herein as containing substantially the referenced compound having a given effect within the formulation or composition, and no further compound with such effect or at most amounts of such compounds which do not exhibit a measurable or relevant effect. The term “about” in the context of a given numeric value or range relates in particular to a value or range that is within 20%, within 10%, or within 5% of the value or range given. As used herein, the term “comprising” also encompasses the term “consisting of”.

The term “isolated” means that the material is substantially free from at least one other component with which it is naturally associated within its original environment. For example, a naturally occurring polynucleotide, polypeptide, or enzyme present in a living animal is not isolated, but the same polynucleotide, polypeptide, or enzyme, separated from some or all of the coexisting materials in the natural system, is isolated. As further example, an isolated nucleic acid, e.g., a DNA or RNA molecule, is one that is not immediately contiguous with the 5′ and 3′ flanking sequences with which it normally is immediately contiguous when present in the naturally occurring genome of the organism from which it is derived. Such polynucleotides could be part of a vector, incorporated into a genome of a cell with an unrelated genetic background (or into the genome of a cell with an essentially similar genetic background, but at a site different from that at which it naturally occurs), or produced by PCR amplification or restriction enzyme digestion, or an RNA molecule produced by in vitro transcription, and/or such polynucleotides, polypeptides, or enzymes could be part of a composition, and still be isolated in that such vector or composition is not part of its natural environment.

“Purified” means that the material is in a relatively pure state, e.g., at least about 90% pure, at least about 95% pure, or at least about 98% or 99% pure. Preferably “purified” means that the material is in a 100% pure state.

A “synthetic” or “artificial” compound is produced by in vitro chemical or enzymatic synthesis. It includes, but is not limited to, variant nucleic acids made with optimal codon usage for host organisms, such as a yeast cell host or other expression hosts of choice or variant protein sequences with amino acid modifications, such as e.g. substitutions, compared to the wildtype protein sequence, e.g. to optimize properties of the polypeptide. A synthetic polypeptide is hence to be understood as a polypeptide that is a synthetic, non-naturally occurring, “man-made” protein sequence. Preferably, a synthetic polypeptide is differing from any naturally occurring polypeptide at the time of the invention in at least one amino acid position.

The term “non-naturally occurring” refers to a (poly)nucleotide, amino acid, (poly)peptide, enzyme, protein, cell, organism, or other material that is not present in its original environment or source, although it may be initially derived from its original environment or source and then reproduced by other means. Such non-naturally occurring (poly)nucleotide, amino acid, (poly)peptide, enzyme, protein, cell, organism, or other material may be structurally and/or functionally similar to or the same as its natural counterpart.

The term “native” (or “wildtype” or “endogenous”) cell or organism and “native” (or wildtype or endogenous) polynucleotide or polypeptide refers to the cell or organism as found in nature and to the polynucleotide or polypeptide in question as found in a cell in its natural form and genetic environment, respectively (i.e., without there being any human intervention).

The term “heterologous” (or exogenous or foreign or recombinant) polypeptide is defined herein as:

(a) a polypeptide that is not native to the host cell. The protein sequence of such a heterologous polypeptide is a synthetic, non-naturally occurring, “man-made” protein sequence;
(b) a polypeptide native to the host cell but structural modifications, e.g., deletions, substitutions, and/or insertions, are included as a result of manipulation of the DNA of the host cell by recombinant DNA techniques to alter the native polypeptide; or
(c) a polypeptide native to the host cell whose expression is quantitatively altered or whose expression is directed from a genomic location different from the native host cell as a result of manipulation of the DNA of the host cell by recombinant DNA techniques, e.g., a stronger promoter.

Descriptions b) and c), above, refer to a sequence in its natural form but not naturally expressed by the cell used for its production. The produced polypeptide is therefore more precisely defined as a “recombinantly expressed endogenous polypeptide”, which is not in contradiction to the above definition but reflects the specific situation that it's not the sequence of a protein being synthetic or manipulated but the way the polypeptide molecule is produced.

Similarly, the term “heterologous” (or exogenous or foreign or recombinant) polynucleotide refers:

(a) to a polynucleotide that is not native to the host cell;
(b) a polynucleotide native to the host cell but structural modifications, e.g., deletions, substitutions, and/or insertions, are included as a result of manipulation of the DNA of the host cell by recombinant DNA techniques to alter the native polynucleotide;
(c) a polynucleotide native to the host cell whose expression is quantitatively altered as a result of manipulation of the regulatory elements of the polynucleotide by recombinant DNA techniques, e.g., a stronger promoter; or
(d) a polynucleotide native to the host cell but integrated not within its natural genetic environment as a result of genetic manipulation by recombinant DNA techniques.

With respect to two or more polynucleotide sequences or two or more amino acid sequences, the term “heterologous” is used to characterize that the two or more polynucleotide sequences or two or more amino acid sequences do not occur naturally in the specific combination with each other.

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

For nucleotide sequences, e.g., consensus sequences, an IUPAC nucleotide nomenclature (Nomenclature Committee of the International Union of Biochemistry (NC-IUB) (1984). “Nomenclature for Incompletely Specified Bases in Nucleic Acid Sequences”.) is used, with the following nucleotide and nucleotide ambiguity definitions, relevant to this invention: A, adenine; C, cytosine; G, guanine; T, thymine; K, guanine or thymine; R, adenine or guanine; W, adenine or thymine; M, adenine or cytosine; Y, cytosine or thymine; D, not a cytosine; N, any nucleotide. In addition, notation “N(3-5)” means that indicated consensus position may have 3 to 5 any (N) nucleotides. For example, a consensus sequence “AWN(4-6)” represents 3 possible variants—with 4, 5, or 6 any nucleotides at the end: AWNNNN, AWNNNNN, AWNNNNNN.

The terms “regulatory element” and “regulatory sequence” are all used interchangeably herein and are to be taken in a broad context to refer to regulatory nucleic acid sequences capable of effecting expression of the sequences to which they are associated, including but not limited thereto, the expression of a polynucleotide encoding a polypeptide. Regulatory elements or regulatory sequences may include any nucleotide sequence having a function or purpose individually and/or within a particular arrangement or grouping of other elements or sequences within the arrangement. Examples of regulatory sequences include, but are not limited to, a leader or signal sequence (such as a 5′-UTR), a start signal, a pro-peptide sequence, a promoter, an enhancer, a silencer, a polyadenylation sequence, a ribosomal binding site (RBS, shine dalgarno sequence), a stop signal, a terminator, a 3′-UTR, and combinations thereof. Regulatory elements or regulatory sequences may be native (i.e. from the same gene) or foreign (i.e. from a different gene) to each other or to a nucleotide sequence to be expressed.

The term “operably linked” means that the described components are in a relationship permitting them to function in their intended manner. For example, a regulatory sequence operably linked to a coding sequence is ligated in such a way that expression of the coding sequence is achieved under condition compatible with the regulatory sequences.

Nucleic acids and polypeptides may be modified to include tags or domains. Tags may be utilized for a variety of purposes, including for detection, purification, solubilization, or immobilization, and may include, for example, biotin, a fluorophore, an epitope, a mating factor, or a regulatory sequence. Domains may be of any size and which provides a desired function (e.g., imparts increased stability, solubility, activity, simplifies purification) and may include, for example, a binding domain, a signal sequence, a promoter sequence, a regulatory sequence, an N-terminal extension, or a C30 terminal extension. Combinations of tags and/or domains may also be utilized.

The term “fusion protein” refers to two or more polypeptides joined together by any means known in the art. These means include chemical synthesis or splicing the encoding nucleic acids by recombinant engineering.

### Methods of Modification of Nucleic Acids to Introduce Changes in the Encoded Protein

Gene Editing

Gene editing or genome editing is a type of genetic engineering in which DNA is inserted, replaced, or removed from a genome and which can be obtained by using a variety of techniques such as “gene shuffling” or “directed evolution” consisting of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547), or with “T-DNA activation” tagging (Hayashi et al. Science (1992) 1350-1353), where the resulting transgenic organisms show dominant phenotypes due to modified expression of genes close to the introduced promoter, or with “TILLING” (Targeted Induced Local Lesions In Genomes) and refers to a mutagenesis technology useful to generate and/or identify nucleic acids encoding proteins with modified expression and/or activity. TILLING also allows selection of organisms carrying such mutant variants. Methods for TILLING are well known in the art (McCallum et al., (2000) Nat Biotechnol 18: 455-457; reviewed by Stemple (2004) Nat Rev Genet 5(2): 145-50). Another technique uses artificially engineered nucleases like Zinc finger nucleases, Transcription Activator-Like Effector Nucleases (TALENs), the CRISPR/Cas system, and engineered meganuclease such as re-engineered homing endonucleases (Esvelt, K M.; Wang, H H. (2013), Mol Syst Biol 9 (1): 641; Tan, W S. et al. (2012), Adv Genet 80: 37-97; Puchta, H.; Fauser, F. (2013), Int. J. Dev. Biol 57: 629-637).

Mutagenesis

DNA and the proteins that they encoded can be modified using various techniques known in molecular biology to generate variant proteins or enzymes with new or altered properties. For example, random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196.

Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793.

Alternatively, modifications, additions or deletions are introduced by error-prone PCR, shuffling, site-directed mutagenesis, assembly PCR, sexual PCR mutagenesis, in vivo mutagenesis (phage-assisted continuous evolution, in vivo continuous evolution), cassette mutagenesis, recursive ensemble mutagenesis, exponential ensemble mutagenesis, site-specific mutagenesis, gene reassembly, gene site saturation mutagenesis (GSSM), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods.

Alternatively, “gene site saturation mutagenesis” or “GSSM” includes a method that uses degenerate oligonucleotide primers to introduce point mutations into a polynucleotide, as described in detail in U.S. Pat. Nos. 6,171,820 and 6,764,835.

Alternatively, Synthetic Ligation Reassembly (SLR) includes methods of ligating oligonucleotide building blocks together non-stochastically (as disclosed in, e.g., U.S. Pat. No. 6,537,776). Alternatively, Tailored multi-site combinatorial assembly (“TMSCA”) is a method of producing a plurality of progeny polynucleotides having different combinations of various mutations at multiple sites by using at least two mutagenic non-overlapping oligonucleotide primers in a single reaction (as described in PCT Pub. No. WO 2009/018449).

Sequence alignments can be generated with a number of software tools, such as:
Needleman and Wunsch algorithm—Needleman, Saul B. & Wunsch, Christian D. (1970). “A general method applicable to the search for similarities in the amino acid sequence of two proteins”. Journal of Molecular Biology. 48 (3): 443-453.

This algorithm is, for example, implemented into the “NEEDLE” program, which performs a global alignment of two sequences. The NEEDLE program, is contained within, for example, the European Molecular Biology Open Software Suite (EMBOSS).
EMBOSS—a collection of various programs: The European Molecular Biology Open Software Suite (EMBOSS), Trends in Genetics 16 (6), 276 (2000).
BLOSUM (BLOcks SUbstitution Matrix)—typically generated on the basis of alignments of conserved regions, e.g. of protein domains (Henikoff S, Henikoff J G: Amino acid substitution matrices from protein blocks. Proceedings of the National Academy of Sciences of the USA. 1992 Nov. 15; 89(22):10915-9). One out of the many BLOSUMs is “BLOSUM62”, which is often the “default” setting for many programs, when aligning protein sequences.
BLAST (Basic Local Alignment Search Tool)—consists of several individual programs (BlastP, BlastN, . . . ) which are mainly used to search for similar sequence in large sequence databases. BLAST programs also create local alignments. Typically used is the “BLAST” interface provided by NCBI (National Center for Biotechnology Information), which is the improved version (“BLAST2”). The “original” BLAST: Altschul, S. F., Gish, W., Miller, W., Myers, E. W. & Lipman, D. J. (1990) “Basic local alignment search tool.” J. Mol. Biol. 215:403-410; BLAST2: Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

Seq A: AAGATACTG length: 9 bases

Seq B: GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “I” symbol in the alignment indicates identical residues (which means bases for DNA or amino acids for proteins). The number of identical residues is 6.

The “−” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

Producing a pairwise alignment which is showing sequence B over its complete length according to the invention consequently results in:

Seq A: GATACTG-
||| |||
Seq B: GAT-CTGA

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the shorter sequence over its complete length)*100. Thus, sequence identity in relation to comparison of two amino acid sequences according to this embodiment is calculated by dividing the number of identical residues by the length of the alignment region which is showing the shorter sequence over its complete length. This value is multiplied with 100 to give “%-identity”. According to the example provided above, %-identity is: (6/8)*100=75%.

Variants of the santalene synthase may have an amino acid sequence which is at least n percent identical to the amino acid sequence of the respective parent polypeptide molecule with n being an integer between 50 and 100, preferably 50, 55, 60, 65, 70, 75, 80, 85, 90, 91, 92, 93, 94, 95, 96, 97, 98 or 99 compared to the full-length polypeptide sequence.

Santalene synthase variants may be defined by their sequence similarity when compared to a parent enzyme. Sequence similarity usually is provided as “% sequence similarity” or “%-similarity”. For calculating sequence similarity in a first step a sequence alignment has to be generated as described above. In a second step, the percent-similarity has to be calculated, whereas percent sequence similarity takes into account that defined sets of amino acids share similar properties, e.g., by their size, by their hydrophobicity, by their charge, or by other characteristics. Herein, the exchange of one amino acid with a similar amino acid is called “conservative mutation”. Enzyme variants comprising conservative mutations appear to have a minimal effect on protein folding resulting in certain enzyme properties being substantially maintained when compared to the enzyme properties of the parent enzyme.

For determination of %-similarity according to this invention the following applies, which is also in accordance with the BLOSUM62 matrix as for example used by the “NEEDLE” program (as referenced above), which is one of the most used amino acids similarity matrix for database searching and sequence alignments.

Amino acid A is similar to amino acids S

Amino acid D is similar to amino acids E; N

Amino acid E is similar to amino acids D; K; Q

Amino acid F is similar to amino acids W; Y

Amino acid H is similar to amino acids N; Y

Amino acid I is similar to amino acids L; M; V

Amino acid K is similar to amino acids E; Q; R

Amino acid L is similar to amino acids I; M; V

Amino acid M is similar to amino acids I; L; V

Amino acid N is similar to amino acids D; H; S

Amino acid Q is similar to amino acids E; K; R

Amino acid R is similar to amino acids K; Q

Amino acid S is similar to amino acids A; N; T

Amino acid T is similar to amino acids S

Amino acid V is similar to amino acids I; L; M

Amino acid W is similar to amino acids F; Y

Amino acid Y is similar to amino acids F; H; W.

Conservative amino acid substitutions may occur over the full length of the sequence of a polypeptide sequence of a functional protein such as an enzyme. In one embodiment, such mutations are not pertaining the functional domains of an enzyme. In one embodiment, conservative mutations are not pertaining the catalytic centres of an enzyme.

Therefore, according to the present description the following calculation of percent-similarity applies: %-similarity=[(identical residues+similar residues)/length of the alignment region which is showing the shorter sequence over its complete length]*100. Thus, sequence similarity in relation to comparison of two amino acid sequences according to this embodiment is calculated by dividing the number of identical residues plus the number of similar residues by the length of the alignment region which is showing the shorter sequence over its complete length. This value is multiplied with 100 to give “%-similarity”.

Variant enzymes comprising conservative mutations which are at least m % similar to the respective parent sequences with m being an integer between 50 and 100, preferably 50, 55, 60, 65, 70, 75, 80, 85, 90, 91, 92, 93, 94, 95, 96, 97, 98 or 99 compared to the full-length polypeptide sequence, are expected to have essentially unchanged enzyme properties, such as enzymatic activity.

“Construct”, “genetic construct” or “expression cassette (used interchangeably) as used herein, is a DNA molecule composed of at least one sequence of interest to be expressed, operably linked to one or more regulatory sequences (at least to a promoter) as described herein. Typically, the expression cassette comprises three elements: a promoter sequence, an open reading frame, and a 3′ untranslated region that, in eukaryotes, usually contains a polyadenylation site. Additional regulatory elements may include transcriptional as well as translational enhancers. An intron sequence may also be added to the 5′ untranslated region (UTR) or in the coding sequence to increase the amount of the mature message that accumulates in the cytosol. The skilled artisan is well aware of the genetic elements that must be present in the expression cassette to be successfully expressed. Preferably, at least part of the DNA or the arrangement of the genetic elements forming the expression cassette is artificial. The expression cassette may be part of a vector or may be integrated into the genome of a host cell and replicated together with the genome of its host cell. The expression cassette is capable of increasing or decreasing the expression of DNA and/or protein of interest.

The term “introduction” or “transformation” as referred to herein encompasses the transfer of an exogenous polynucleotide into a host cell, irrespective of the method used for transfer. That is, the term “transformation” as used herein is independent from vector, shuttle system, or host cell, and it not only relates to the polynucleotide transfer method of transformation as known in the art (cf., for example, Sambrook, J. et al. (1989) Molecular Cloning: A Laboratory Manual, 2nd Ed., Cold Spring Harbor Laboratory Press, Cold Spring Harbor, N.Y.), but it encompasses any further kind polynucleotide transfer methods such as, but not limited to, transduction or transfection.

The term “recombinant organism” refers to a eukaryotic organism (yeast, fungus, alga, plant, animal) or to a prokaryotic microorganism (e.g., bacteria) which has been genetically altered, modified or engineered such that it exhibits an altered, modified or different genotype as compared to the wild-type organism which it was derived from. Preferably, the “recombinant organism” comprises an exogenous nucleic acid. “Recombinant organism”, “genetically modified organism” and “transgenic organism” are used herein interchangeably. The exogenous nucleic acid can be located on an extrachromosomal piece of DNA (such as plasmids) or can be integrated in the chromosomal DNA of the organism. In the case of a recombinant eukaryotic organism, it is understood as meaning that the nucleic acid(s) used are not present in, or originating from, the genome of said organism, or are present in the genome of said organism but not at their natural locus in the genome of said organism, it being possible for the nucleic acids to be expressed under the regulation of one or more endogenous and/or exogenous regulatory element.

“Host cells” may be any cell selected from bacterial cells, yeast cells, fungal, algal or cyanobacterial cells, non-human animal or mammalian cells, or plant cells. The skilled artisan is well aware of the genetic elements that must be present on the genetic construct to successfully transform, select and propagate host cells containing the sequence of interest. Host cells may be selected from any of these organisms:

### Bacteria

gram positive: Bacillus, Streptomyces
   Useful gram positive bacteria include, but are not limited to, a Bacillus cell, e.g., Bacillus alkalophius, Bacillus amyloliquefaciens, Bacillus brevis, Bacillus circulans, Bacillus clausii, Bacillus coagulans, Bacillus firmus, Bacillus Jautus, Bacillus lentus, Bacillus licheniformis, Bacillus megaterium, Bacillus pumilus, Bacillus stearothermophilus, Bacillus subtilis, and Bacillus thuringiensis. Most preferred, the prokaryote is a Bacillus cell, preferably, a Bacillus cell of Bacillus subtilis, Bacillus pumilus, Bacillus licheniformis, or Bacillus lentus.
   Some other preferred bacteria include strains of the order Actinomycetales, preferably, Streptomyces, preferably Streptomyces spheroides (ATTC 23965), Streptomyces thermoviolaceus (IFO 12382), Streptomyces lividans or Streptomyces murinus or Streptoverticillum verticillium ssp. verticillium. Other preferred bacteria include Rhodobacter sphaeroides, Rhodomonas palustri, Streptococcus lactis. Further preferred bacteria include strains belonging to Myxococcus, e.g., M. virescens.
gram negative: E. coli, Pseudomonas
   Preferred gram negative bacteria are Escherichia coli and Pseudomonas sp., preferably, Pseudomonas purrocinia (ATCC 15958) or Pseudomonas fluorescens (NRRL B-11).

### Fungi

Aspergillus, Fusarium, Trichoderma
   The microorganism may be a fungal cell. “Fungi” as used herein includes the phyla Ascomycota, Basidiomycota, Chytridiomycota, and Zygomycota as well as the Oomycota and Deuteromycotina and all mitosporic fungi. Representative groups of Ascomycota include, e.g., Neurospora, Eupenicillium (=Penicillium), Emericella (=Aspergillus), Eurotium (=Aspergillus), and the true yeasts listed below. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g. Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.
   Some preferred fungi include strains belonging to the subdivision Deuteromycotina, class Hyphomycetes, e.g., Fusarium, Humicola, Tricoderma, Myrothecium, Verticillum, Arthromyces, Caldariomyces, Ulocladium, Embellisia, Cladosporium or Dreschlera, in particular Fusarium oxysporum (DSM 2672), Humicola insolens, Trichoderma resii, Myrothecium verru cana (IFO 6113), Verticillum alboatrum, Verticillum dahlie, Arthromyces ramosus (FERM P-7754), Caldariomyces fumago, Ulocladium chartarum, Embellisia alli or Dreschlera halodes.
   Other preferred fungi include strains belonging to the subdivision Basidiomycotina, class Basidiomycetes, e.g. Coprinus, Phanerochaete, Coriolus or Trametes, in particular Coprinus cinereus f. microsporus (IFO 8371), Coprinus macrorhizus, Phanerochaete chrysosporium (e.g. NA-12) or Trametes (previously called Polyporus), e.g. T. versicolor (e.g. PR4 28-A).
   Further preferred fungi include strains belonging to the subdivision Zygomycotina, class Mycoraceae, e.g. Rhizopus or Mucor, in particular Mucor hiemalis.

### Yeast

Pichia
Saccharomyces
   The fungal host cell may be a yeast cell. “Yeast” as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g. genera Kluyveromyces, Pichia, and Saccharomyces). The basidiosporogenous yeasts include the genera Leucosporidim, Rhodosporidium, Sporidiobolus, Filobasidium, and Filobasidiella. Yeasts belonging to the Fungi Imperfecti are divided into two families, Sporobolomycetaceae (e.g., genera Sporobolomyces and Bullera) and Cryptococcaceae (e.g. genus Candida).

### Eukaryotes

Non-human animal, non-human mammal, avian, reptilian, insect, plant, yeast, fungi

The term “santalene synthase” is used herein for polypeptides having catalytic activity in the formation of santalene and santalene-like terpenes like α-santalene, β-santalene, trans-α-bergamotene and epi-β-santalene from farnesyl diphosphate, and for other moieties comprising such a polypeptide. Examples of such other moieties include complexes of said polypeptide with one or more other polypeptides, fusion proteins of comprising a santalene synthase polypeptide fused to a peptide or protein tag sequence, other complexes of said polypeptides (e.g. metalloprotein complexes), macromolecular compounds comprising said polypeptide and another organic moiety, said polypeptide bound to a support material, etc. The santalene synthase can be provided in its natural environment, i.e. within a cell in which it has been produced, or in the medium into which it has been excreted by the cell producing it, It can also be provided separate from the source that has produced the polypeptide and can be manipulated by attachment to a carrier, labelled with a labelling moiety, and the like.

The activity and product profile of santalene synthases can be measured with known methods, for example as disclosed in the international patent application published as WO2018160066.

In the following, the terms “synthetic santalene synthase” and “improved santalene synthase” are used interchangeably to refer to a santalene synthase of synthetic sequence that under typical conditions produces beta-santalene in excess of alpha-santalene or increased alpha-santalene amounts compared the wildtype santalene synthase.

“Improved alpha santalene synthases” refers hence to those synthetic santalene synthases that have an increased alpha santalene production compared to their counterpart from nature that under typical conditions. “Improved beta santalene synthase” refers to a santalene synthase of synthetic sequence that under typical conditions produces beta-santalene in excess of alpha-santalene.

The term “in excess” is used interchangeably with the term “surplus” and is to be understood that more of the first named substance is present than of the substance named second. A in excess of B hence means that more of substance A is present that on substance B, on the same basis which may be molar or weight or percentage.

In the conversion of Farnesyl pyrophosphate to terpene product, the diphosphate is cleaved to generate a reactive carbocation transition state, leading to a series of potential reactions such as hydride shifts and cyclizations. Residues that are involved in favouring some potential transition state over others can therefore affect the final product ratios of the possible products. The main products of known santalene synthases are primarily alpha-Santalene, bergamotene and/or beta santalene.

Santalene synthases are enzymes of the terpene synthase family and due to the multitude of products produced from the same substrate are classified as belonging to the enzyme classes EC4.2.3.81, EC4.2.3.82 and/or EC4.2.3.83, or EC4.2.3.50—enzymes of the later class use (2Z,6Z)-farnesyl diphosphate as a substrate instead of (2E,6E)-farnesyl diphosphate. They comprise an N-terminal PFAM domain PF01397 and a C-terminal PFAM domain PF03936 (analysed using version 32.0 of PFAM, for PFAM details see “The Pfam protein families database in 2019: S. EI-Gebali, J. Mistry, A. Bateman, S. R. Eddy, A. Luciani, S. C. Potter, M. Qureshi, L. J. Richardson, G. A. Salazar, A. Smart, E. L. L. Sonnhammer, L. Hirsh, L. Paladin, D. Piovesan, S. C. E. Tosatto, R. D. Finn Nucleic Acids Research (2019) and http://pfam.xfam.org/) that comprises the active site and metal binding sites. They require a divalent cation as a co-factor usually magnesium or manganese. In their functional state they typically have three Mg2+ ions coordinated by two metal binding sites that are rich in Aspartates. One of these is termed the DDxxD motif, wherein this is a sequence of two Aspartates, followed by any amino acid, followed by another variable amino acid, preferably a Phenylalanine or Tyrosine, more preferably a Tyrosine, and followed by a further Aspartate. The second metal binding site is termed NSE/DTE triad. This is a sequence of amino acids starting with Asparagine or Aspartate, followed by a second Aspartate, followed by two variable amino acids, followed by a Serine or Threonine, followed again by one or two variable amino acids, followed by a Lysine or Arginine, followed optionally by a variable amino acid and ending with an Aspartate or Glutamate residue. In these motifs, the variable amino acids are preferably those that allow the defined amino acids of the motif to assume the tertiary structure need for metal ion binding, typically magnesium binding.

One of these conserved binding sites coordinating the magnesium ions, the DDxxD motif, is located in an alpha helix. In the santalene synthase from Cinnamomum camphora known as CiCaSSy (provided as SEQ ID NO: 1) this alpha helix stretches from the Proline at position 278 or just after this to the Aspartate at position 302 of SEQ ID NO 1 and is named Helix D. In other santalene synthases there are equivalent alpha helices comprising the DDxxD motif present, albeit their naming may be different, yet the helix always impinges the active site directly. In the following any reference to Helix D is referring to the alpha helix of a given santalene synthase comprising the DDxxD motif, at the positions corresponding to the amino acid positions of 298 to 302 of SEQ ID NO 1, irrespective if the helix may be identified with the letter D or differently in the respective protein sequence. Due to the high conservation of the DDxxD motif and other conserved residues and structural features, these helices are known in the art and can be identified in new sequences of santalene synthases easily.

The inventors realised that Helix D is crucial for the product profile of a santalene synthase yet changing it could unduly disturb the enzyme structure in sensitive areas of the active site and/or endanger the magnesium ion binding required for the enzyme action.

The inventors found that a change in product profile of the enzyme can be realised by a more subtle change. In santalene synthases, Helix D is preceded by another alpha helix. In CiCaSSy this is termed Helix C and stretches from position 263 to position 272 in SEQ ID NO: 1. Some predictions extend this alpha-helix to position 276, yet the core is from positions 263 to 272. There is an Arginine residue at the start of the helix in position 263 of SEQ ID NO: 1 which is part of Arginine-Aspartate-Arginine triad found in positions 261 to 263 of SEQ ID NO: 1. This triad contains at the N-terminal end an Arginine residue that is conserved in santalene synthases.

Helix C interacts with Helix D on their facing sides. Particular relevant amino acid positions of Helix D are in the area corresponding to position 291 of SEQ IDN O: 1Further positions with possible side chain interactions to the side chains of the amino acids of Helix C are upstream at positions 287 and 288, Isoleucine and Threonine, respectively, in SEQ IDNO: 1, 2 and 3 and downstream at positions 294 and 295, Methionine and Threonine, respectively, in SEQ ID NO: 1, 2 and 3.

The inventors found that manipulation of Helix C provides the enzyme with more flexibility that will affect the products produced, while at the same time not disturbing unduly the enzyme structure or the magnesium binding or the substrate binding of the enzyme in a negative fashion. They found that from the primary structure, many santalene synthases seem to be amenable to the desired changes in principle and choose CiCaSSy (SEQ ID NO: 1) to demonstrate the inventive effect. CiCaSSy shares in Helix C elements with santalene synthases with a relatively high production of beta-santalene albeit still less than the alpha santalene produced, which is also CiCaSSy's product profile with respect to these two santalenes. Examples of such known enzymes next to CiCaSSy are SaSSy (SEQ ID NO: 4) SaSSy14 (SEQ ID NO: 5), SspiSSy (SEQ ID NO: 6) or SauSSy (SEQ ID NO: 7) or SaSSy134 (SEQ ID NO: 9). Yet CiCaSSy also shares elements with santalene synthases that are low producers of beta-santalene and strong alpha-santalene producers like ClaSSy (SEQ ID NO: 8). Due to this intermediate position between these groups CiCaSSy was chosen as the starting point for manipulating Helix C in order to affect the flexibility of the enzyme structure, for example of Helix D and other downstream parts in a positive manner.

After in depth study, the residue 267 of CiCaSSy was chosen for mutation. This residue is expected to interact with the face of Helix D with its side chain (see FIG. 3). Neighbouring to this residue, CiCaSSy has some less common amino acids compared to other santalene synthases that were expected to make it more amenable to result in changes of the product profile. At the position corresponding to the Asparagine 267 (termed N267) of SEQ ID NO 1, many other santalene synthases have either a Serine or a Leucine (see FIG. 1 alignment). Yet these santalene synthases with Serine or Leucine at this position have the described downside in their product profile, similar to the unmutated CiCaSSy of SEQ ID NO: 1. However, the inventors realized that the surrounding of N267 in SEQ ID NO: 1 is so favourable that the inventors chose to replace that unusual Asparagine at position 267 with Serine and Leucine, although these are found at the corresponding location in other santalene synthases of known lacking performance.

The resulting synthetic protein sequence for the improved santalene synthases named N267S and N267L are given in SEQ ID NO:2 and SEQ ID NO: 3, respectively. Surprisingly, the reversion to a more common amino acid at this position resulted in a change in spatial flexibility of the catalytic part of the enzyme for example of the two neighboured a helices and novel, favourable change in product profile. Further, this favourable change in product profile could also be achieved with other, skillful replacements for the position corresponding to 267 of SEQ ID NO: 1, for example with Glycine or Alanine as shown herein below.

The DNA sequences encoding wildtype CiCaSSy, N267S and N267L are listed as SEQ ID NO: 10, 11 and 12, respectively.

Additional synthetic protein sequences carrying the Serine at a position corresponding to position 267 of SEQ ID NO: 1 are shown as SEQ ID NO: 13 to 20, and additional improved protein sequences carrying the Leucine at a position corresponding to position 267 of SEQ ID NO: 1 are shown as SEQ ID NO: 21 to 28.

In one embodiment the invention hence refers to a synthetic beta santalene synthase producing beta-santalene in excess of alpha-santalene from farnesyl pyrophosphate under conditions that typically result in the production of both these santalenes, albeit the known santalene synthases typically produce alpha-santalene in excess of beta-santalene under such conditions, wherein the inventive synthetic beta santalene synthase is characterized by the fact that the flexibility of the tertiary structures that correspond to the alpha helix stretching from amino acid positions 272 to position 291, preferably to position 284, of SEQ ID NO: 1, is increased compared to the same tertiary structure in a naturally occurring santalene synthase that is producing a surplus of alpha-santalene over beta-santalene. The flexibility can be determined for example by root mean square fluctuation analysis using simulations for 500 ns in the identical conditions with the settings pH 8.0, 300 K, 1 atm, water environment, ions present without substrate, and evaluation for each enzyme structure on the last 450 ns of simulation, and wherein the calculations were performed by the gmx rmsf tool of the GROMACS software version 2018 after having performed a structural superimposition of the protein structure for each trajectory frame using gmx trjconv and using the protein Cα of the equilibrated system as a reference.

In one embodiment, the polypeptide of the invention is a synthetic polypeptide with the enzymatic function of a beta santalene synthase and means to increase the flexibility of Helix D, preferably the flexibility of the tertiary structures that correspond to the alpha helix stretching from amino acid positions corresponding to the positions 272 to position 291 in SEQ ID NO: 1, compared to its naturally occurring counterparts, and further characterized by a production of beta santalene in excess of alpha santalene from FPP under conditions suitable for beta santalene production.

In one aspect of the invention the flexibility of the tertiary structures that correspond to the stretch from amino acid positions 272 to position 291 preferably to position 284, of SEQ ID NO: 1, is increased compared to the same tertiary structure in a naturally occurring santalene synthase that is producing a surplus of alpha-santalene over beta-santalene wherein the flexibility is determined by root mean square fluctuation analysis using simulations for 500 ns in the identical conditions with the settings pH 8.0, 300 K, 1 atm, water environment, ions present without substrate, and evaluation for each enzyme structure on the last 450 ns of simulation, and wherein the calculations were performed by the gmx rmsf tool of the GROMACS software version 2018 after having performed a structural superimposition of the protein structure for each trajectory frame using gmx trjconv and using the protein Cα of the equilibrated system as a reference. The increase in flexibility is at least 5%, preferably at least 10%, more preferably at least 15% compared to the flexibility of the corresponding tertiary structure of a naturally occurring santalene synthase that is producing a surplus of alpha-santalene over beta-santalene. In a further embodiment the position corresponding to position 267 of SEQ ID NO: 1 is filled with a Serine, Leucine, Threonine, Cysteine, Isoleucine, Valine, Tryptophan, Glycine or an Alanine, preferably Serine, Threonine, Tryptophan, Glycine, Alanine or Leucine, more preferably Serine, Glycine, Alanine or Leucine. In another aspect of the invention the synthetic santalene synthase further comprises two aspartate rich motifs for binding Mg2+, preferably the DDxxD motif and the NSE/DTE triad.

In one embodiment, the improved beta santalene synthases comprise a stretch of amino acids from Arginine corresponding to position 261 of SEQ ID NO: 1 (R261) to two aspartic acid residues corresponding to positions 298 and 299 of SEQ ID NO: 1 (D298 and D299), followed by two amino acids, preferably the second of these being a Tyrosine, and followed by a third aspartic acid corresponding to position 302 of SEQ ID NO: 1 (D302), wherein these five amino acids preferably are involved in metal binding of the enzyme, and preferably the position corresponding to position 267 of SEQ ID NO: 1 is filled with a Serine, Leucine, Threonine, Cysteine, Isoleucine, Valine, Tryptophan, Glycine or an Alanine, preferably Serine, Threonine, Tryptophan, Glycine, Alanine or Leucine, more preferably Serine, Glycine, Alanine or Leucine. In a preferred embodiment, the synthetic santalene synthase comprises such a stretch, wherein further said stretch starting with an Arginine corresponding to R261 of SEQ ID NO: 1 and ending with an Aspartate corresponding to D302 of SEQ ID NO: 1 and in addition has in order of increasing preference at least 50%, 60%, 65%, 70%, 75%, 80% 85%, 86%, 87%, 88%, 89%, 90%, 91%, 92%, 93%, 94%, 95%, or 97% sequence identity over the full length of the amino acids 261 to 302 of SEQ ID NO: SEQ ID NO: 2, 3, 13 to 53, preferably of those from SEQ ID NO: 2, 3, 14 to 17, 21 to 52, wherein more preferably all strongly conserved amino acids in this stretch as depicted in FIG. 1 are present in the stretch.

In one aspect of the invention, the improved santalene synthases of the invention and useful in the methods and host cells of the invention carry a R(R/K)xxxxxxxxW motif (Arginine followed by an Arginine or Lysine, then eight amino acids of any type, then an Arginine, see SEQ ID NO: 55), preferably the motif RRxxxxxxxxW (RRX8W, see SEQ ID NO: 54), close to their N-terminal start. In one embodiment the RRX8W motif starts at the position corresponding to position 7 in SEQ ID NO: 2, 3, 29, 57 or 58 and ends at the position corresponding to position 17 of SEQ ID NO: 2, 3 or 29. In another embodiment, the RRX8W motif found in the improved santalene synthases of the invention and useful in the methods and host cells of the invention have in positions corresponding to positions 7 to 17 of SEQ ID NO: 2, 3, 29, 57 or 58 identical amino acids to those of SEQ ID NO: 2, 3, 29, 57 or 58 in the following positions of SEQ ID NO: 2, 3 or 29: 7, 8 and 12 to 17.

In a further embodiment, the improved santalene synthases of the invention and useful in the methods and host cells of the invention holds an RRX8W motif close to their N-terminal start that is at least 80 or 90% identical to the RRX8W motif as found in SEQ ID NO: 2, 3 or 29. In another aspect this motif in the improved santalene synthases of the invention and useful in the methods and host cells of the invention is identical to the RRX8W motif of SEQ ID NO: 2, 3 or 29.

In one aspect of the invention, the improved santalene synthases of the invention and useful in the methods and host cells of the invention comprise a PFAM domain PF01397 “Terpene_synth” and a C-terminal PFAM domain PF03936 “Terpene_synth_C”.

In another aspect of the invention, the improved santalene synthases of the invention and useful in the methods and host cells of the invention comprise the following features identified by the InterPro software:

Domains “Terpene synthase, metal-binding domain” IPR005630, “Terpene cyclase-like 1, C-terminal domain” IPR034741 and “Terpene synthase, N-terminal domain” IPR001906 and the homologous superfamilies “Isoprenoid synthase domain superfamily” IPR008949, “Terpenoid cyclases/protein prenyltransferase alpha-alpha toroid” IPR008930 and “Terpene synthase, N-terminal domain superfamily” IPR036965.

As demonstrated, only one or several amino acid changes are necessary in the key area of Helix C to provide for the desired effect of an improved product profile. Due to the shortness of the Helix C area, one or several changes quickly results in relatively large differences in the sequence identity of two sequences for the Helix C area.

A further preferred embodiment relates to a synthetic santalene synthase improved over the wildtype enzyme so that it is producing beta-santalene in excess of alpha-santalene from famesyl pyrophosphate, wherein the santalene synthase has at least 50%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity over the full length of the amino acid positions 261 to 278 of SEQ ID NO: 2, 3 or 29, preferably to position 261 to position 272 of SEQ ID NO: 2, 3 or 29, using an Arginine residue that corresponds to the Arginine at position 261 of SEQ ID No. 2 or 3 and a Proline residue that correspond to the Proline at position 278 of SEQ ID NO: 2, 3, 29, 57 or 58 to align the two protein sequences for the sequence identity determination, and more preferably the position corresponding to position 267 of SEQ ID NO: 2, 3, 29, 57 or 58 is filled with a Serine, Leucine, Threonine, Cysteine, Isoleucine, Valine, Tryptophan, Glycine or an Alanine, preferably Serine, Threonine, Tryptophan, Glycine, Alanine or Leucine, more preferably Serine, Glycine, Alanine or Leucine, and the position corresponding to position 291 of SEQ ID NO: SEQ ID NO: 2, 3, 29, 57 or 58 is filed with an amino acid other than Histidine or Leucine; preferably this position is filled with any of these amino acids: Isoleucine, Valine, Serine, Cysteine, Phenylalanine or Threonine. In one aspect of the invention said synthetic beta santalene synthase is producing beta-santalene and alpha-santalene in a ratio that is equal to or greater than 1, preferably at least 1.1 and more preferably at least 1.2 and even more preferably 1.3 under conditions suitable for the production of these santalenes.

Another aspect of the invention is to a synthetic beta santalene synthase producing beta-santalene in excess and of alpha-santalene from farnesyl pyrophosphate, wherein the santalene synthase has at least 50%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to the amino acid positions 261 to 302 of SEQ ID NO: 2, 3, 29 to 40, 57 or 58, wherein the position corresponding to position 261 of SEQ ID No. 2 or 3 is an Arginine residue that corresponds to the Arginine at position 261 of SEQ ID No. 2 or 3 and three Aspartate residues are found that at positions that correspond to the Aspartates at positions 298, 299 and 302 of SEQ ID NO: 2 or 3 or 29 to 40, and wherein said synthetic beta santalene synthase is producing beta-santalene and alpha-santalene in a ratio that is equal to or greater than 1, preferably at least 1.1 and more preferably at least 1.2 and even more preferably 1.3 under conditions suitable for the production of these santalenes.

In a preferred embodiment the improved beta santalene synthase the position corresponding to position 267 of SEQ ID NO: 1 is filled with a Serine, Leucine, Threonine, Cysteine, Isoleucine, Valine, Tryptophan, Glycine or an Alanine, preferably Serine, Glycine, Alanine or Threonine and the position corresponding to the position 282 of SEQ ID NO: 1 is filled with an amino acid that has a polar uncharged side chain or a positively charged side chain, preferably with a Glutamine or Asparagine or Arginine or Lysine.

In another preferred embodiment, in the improved santalene synthase the position corresponding to position 267 of SEQ ID NO: 1 is filled with a Serine, Leucine, Threonine, Cysteine, Isoleucine, Valine, Tryptophan, Glycine or an Alanine, preferably Serine, Threonine, Tryptophan, Glycine, Alanine or Leucine, more preferably Serine, Glycine, Alanine or Leucine and it also has the following amino acids at the position corresponding to the position in SEQ ID NO 1 provided in brackets behind the name of the amino acid in the following: An Arginine (261), Aspartate or Asparagine (262), Arginine or Asparagine (263), Leucine or Isoleucine or Valine or Methionine (264) Leucine or Isoleucine or Valine or Methionine (265), Glutamic Acid or Glutamine (266), Histidine or Tyrosine (268) and Glutamine or Arginine or Lysine (282).

More preferably these are the following amino acids at the position corresponding to the position in SEQ ID NO 1 provided in brackets: Arginine(261), Aspartate (262), Arginine (263), Leucine (264) Leucine (265), Glutamic Acid (266), Histidine (268), Leucine (269), Phenylalanine (270) and Glutamine or Arginine (282).

In one aspect of the invention, in addition to the defined amino acids as in previous paragraph, the improved beta santalene synthases of the invention the position corresponding to position 291 of SEQ ID NO: SEQ ID NO: 2, 3, 29, 57 or 58 is filed with an amino acid other than Histidine or Leucine, preferably this position is filled with any of these amino acids: Isoleucine, Valine, Serine, Cysteine, Phenylalanine or Threonine.

In yet another preferred embodiment, the improved santalene synthases comprise in addition a Serine or Threonine, preferably Serine, at the position that corresponds to position 271 of SEQ ID NO: 1 and an Alanine, Isoleucine, Valine or Cysteine, preferably an Alanine at the position that corresponds to position 273 of SEQ ID NO: 1.

Further preferably the improved santalene synthases are those that carry in the position corresponding to position 267 of SEQ ID NO: 1 a Serine, Leucine, Threonine, Cysteine, Isoleucine, Valine, Tryptophan, Glycine or an Alanine, preferably Serine, Threonine, Glycine, Alanine or Leucine, more preferably Serine, Glycine, Alanine or Leucine, and in addition the positions corresponding to positions in SEQ ID NO: 1 are filled with the amino acids listed for the corresponding position of SEQ ID NO: 1 in Table A, B or C below.

TABLE A

| Position in | |
|---|---|
| SEQ ID NO: 1_ | Amino acid(s) allowed |
| 271 | Serine or Alanine, preferably Serine |
| 272 | Isoleucine, Valine, Methionine or Leucine, preferably |
| | Isoleucine |
| 273 | Alanine or Glycine, preferably Alanine |
| 274 | Valine or Methionine or Isoleucine or Leucine, preferably |
| | Valine |
| 275 | Valine or Methionine or Isoleucine or Leucine, preferably |
| | Valine |
| 276 | Valine or Methionine, preferably Valine |
| 277 | Glutamate |
| 278 | Proline |
| 279 | Glutamine or Asparagine or Lysine, preferably Glutamine |
| 280 | Tyrosine or Phenylalanine, preferably Tyrosine |
| 281 | Serine or Lysine, preferably Serine |
| 282 | Glutamine or Asparagine or Arginine or Lysine or Serine |
| | or Leucine or Threonine, preferably Glutamine or |
| | Arginine or Lysine |
| 283 | Cysteine or Alanine or Glycine, preferably Cysteine |
| 284 | Arginine |
| 285 | Valine or Methionine or Isoleucine or Leucine, preferably |
| | Valine |
| 286 | Alanine or Glycine or Valine, preferably Alanine |
| 287 | Isoleucine Valine, Methionine or Leucine, preferably |
| | Isoleucine |
| 288 | Threonine or Valine or Serine, preferably Threonine |
| 289 | Lysine |
| 290 | Alanine or Isoleucine, preferably Alanine |
| 291 | Isoleucine, Valine, Leucine, Cysteine, Serine, Methionine, |
| | Phenylalanine or Threonine, preferably Isoleucine, |
| | Phenylalanine, Valine, Cysteine, Serine or Threonine |
| 292 | Valine, Glycine, Methionine or Isoleucine, preferably |
| | Valine |
| 293 | Leucine or Valine, preferably Leucine |
| 294 | Methionine, Isoleucine or Valine, preferably Methionine |
| 295 | Threonine or Serine, preferably Threonine |
| 296 | Alanine, Valine or Leucine, preferably Alanine |
| 297 | Methionine or Valine, preferably Methionine |
| 298 | Aspartate |
| 299 | Aspartate |
| 300 | Phenylalanine or Tyrosine or Alanine or Isoleucine or |
| | Glycine or Threonine, preferably Phenylalanine |
| 301 | Tyrosine or Phenylalanine, preferably Tyrosine |
| 302 | Aspartate |

TABLE B_

| Position in | |
|---|---|
| SEQ ID NO: 1 | Amino acid(s) allowed |
| 271 | Serine |
| 272 | Isoleucine Valine, Methionine or Leucine, preferably |
| | Isoleucine |
| 273 | Alanine |
| 274 | Valine |
| 275 | Valine |
| 276 | Valine |
| 277 | Glutamate |
| 278 | Proline |
| 279 | Glutamine |
| 280 | Tyrosine |
| 281 | Serine |
| 282 | Glutamine or Arginine or Lysine |
| 283 | Cysteine |
| 284 | Arginine |
| 285 | Valine |
| 286 | Alanine |
| 287 | Isoleucine |
| 288 | Threonine |
| 289 | Lysine |
| 290 | Alanine |
| 291 | Isoleucine, Valine, Serine, Cysteine, Phenylalanine or |
| | Threonine |
| 292 | Valine |
| 293 | Leucine |
| 294 | Methionine |
| 295 | Threonine |
| 296 | Alanine |
| 297 | Methionine |
| 298 | Aspartate |
| 299 | Aspartate |
| 300 | Phenylalanine or Tyrosine or Alanine, or Glycine or |
| | Threonine, preferably Phenylalanine |
| 301 | Tyrosine or Phenylalanine, preferably Tyrosine |
| 302 | Aspartate |

TABLE C_

| Position in | |
|---|---|
| SEQ ID | |
| NO: 1 | Amino acid(s) allowed |
| 271 | Serine |
| 272 | Isoleucine |
| 273 | Alanine |
| 274 | Valine |
| 275 | Valine |
| 276 | Valine |
| 277 | Glutamate |
| 278 | Proline |
| 279 | Glutamine |
| 280 | Tyrosine |
| 281 | Serine |
| 282 | Glutamine |
| 283 | Cysteine |
| 284 | Arginine |
| 285 | Valine |
| 286 | Alanine |
| 287 | Isoleucine |
| 288 | Threonine |
| 289 | Lysine |
| 290 | Alanine |
| 291 | Isoleucine, Serine, Cysteine, |
| | Valine, Phenylalanine or |
| | Threonine |
| 292 | Valine |
| 293 | Leucine |
| 294 | Methionine |
| 295 | Threonine |
| 296 | Alanine |
| 297 | Methionine |
| 298 | Aspartate |
| 299 | Aspartate |
| 300 | Phenylalanine |
| 301 | Tyrosine |
| 302 | Aspartate |

The Aspartate at position 298 of SEQ ID NO: 1 marks the start of the DDXXD motif in SEQ IDNO: 1.

In another preferred embodiment, the improved santalene synthases comprise a Histidine at the position that corresponds to position 268 of SEQ ID NO: 1, a Leucine at the position that corresponds to position 269 of SEQ ID NO: 1 and a Phenylalanine at the position that corresponds to position 270 of SEQ ID NO: 1, and preferably the position corresponding to position 267 of SEQ ID NO: 1 is filled with a Serine, Leucine, Threonine, Cysteine, Isoleucine, Valine, Tryptophan, Glycine or an Alanine, preferably Serine, Threonine, Tryptophan, Glycine, Alanine or Leucine, more preferably Serine or Leucine. More preferably, the improved santalene synthase also comprises the amino acids listed in tables A, B or C at the positions corresponding to the positions listed in the tables A, B or C for SEQ ID NO: 1.

In a preferred embodiment the improved beta santalene synthases have at the position corresponding to position 291 of SEQ ID NO: 1 another amino acid than a Histidine, Glycine or Leucine.

The inventors applied a further approach to increase the flexibility around Helix C and Helix D. The position 291 in SEQ ID NO: 1, 2, 3, 29, 57 or 58 is the position that is part of the Helix D facing Helix C. In the wildtype CiCassy of SEQ ID NO: 1, the position is filled with an Isoleucine. Surprisingly, the inventors found that replacing the Isoleucine at position 291 of SEQ ID NO: 1 with a Threonine, Serine, Valine, Phenylalanine or Cysteine has a positive effect on the beta-santalene to alpha-santalene ratio, while maintaining higher alpha-santalene levels than in the N267S or N267L mutant. In another aspect of the invention the synthetic beta santalene synthase with Threonine, Serine, Methionine, Valine, Phenylalanine or Cysteine, preferably Threonine, Serine, Valine, Phenylalanine or Cysteine at the position corresponding to position 291 in SEQ ID NO: 1 further comprises two aspartate rich motifs for binding Mg2+, preferably the DDxxD motif and the NSE/DTE triad.

Further, the inventors created a synthetic santalene sequence with the amino acid at the position corresponding to position 291 of SEQ ID NO: 1 was replaced with a Leucine, and the alpha-santalene production was increased compared to the one of SEQ ID NO: 1.

Yet another aspect of the invention relates to a synthetic santalene synthase with the favourable mutations at the positions corresponding to 267 and/or 291 of SEQ ID NO: 1 wherein the santalene synthase comprises the Aspartate rich motif for binding Mg2+, DDxxD, with a Tyrosine or Phenylalanine at the fourth position, more preferably the binding motif has the sequence starting from the N-terminal end of two Aspartates, Phenylalanine, Tyrosine and followed by a further Aspartate.

Further to the preferred amino acid replacing Isoleucine at the position corresponding to position 291 of SEQ ID NO: 1, the improved santalene synthases have in a preferred embodiment at the position corresponding to position 287 Isoleucine or Leucine, preferably Isoleucine, and at the position corresponding to position 288 in SEQ ID NO: 1 Threonine, Serine or Valine, preferably Threonine or Serine, more preferably Threonine. Furthermore one preferred aspect of the invention relates to an improved santalene synthase with an Alanine at the position corresponding to position 286 of SEQ ID NO: 1, Isoleucine at the position corresponding to position 287 of SEQ ID NO: 1, Threonine at the position corresponding to position 288 of SEQ ID NO: 1, Lysine at the position corresponding to position 289 of SEQ ID NO: 1, Alanine at the position corresponding to position 290 of SEQ ID NO: 1.

In addition to the preferred amino acid replacing Isoleucine at the position corresponding to position 291 of SEQ ID NO: 1, the improved santalene synthases have in a preferred embodiment at the position corresponding to position 294 in SEQ ID NO: 1 a Methionine or Leucine or Glutamic Acid residue, preferably a Methionine or a Glutamic Acid residue, more preferably a Methionine.

One aspect of the invention relates to a synthetic beta santalene synthase producing from farnesyl pyrophosphate beta-santalene in excess of alpha-santalene, wherein the santalene synthase has an amino acid sequence at least 50% identical to SEQ ID NO: 1 and has in the amino acid position corresponding to:
a. position 267 of SEQ ID NO: 1 any of the following amino acids:
   Serine, Leucine, Threonine, Cysteine, Isoleucine, Valine, Tryptophan, Glycine or Alanine; or
b. position 291 of SEQ ID NO: 1 any of the following amino acids:
   Threonine, Cysteine, Serine, Phenylalanine or Valine; or
c. a combination of a. and b. above; or
d. position 267 of SEQ ID NO: 1 an Asparagine and the position corresponding to position 291 of SEQ ID NO: 1 is any of the following amino acids:
   Threonine, Cysteine, Serine, Phenylalanine or Valine; or
e. position 267 of SEQ ID NO: 1 any of the following amino acids:
   Serine, Leucine, Threonine, Cysteine, Isoleucine, Valine, Tryptophan, Glycine or Alanine and the position corresponding to position 291 of SEQ ID NO: 1 is an Isoleucine.

In one other aspect, the invention relates hence to a synthetic beta santalene synthase producing beta-santalene in excess of alpha-santalene from farnesyl pyrophosphate, wherein the santalene synthase has an amino acid sequence at least 60% identical to SEQ ID NO: 1 and has in the amino acid position corresponding to a) position 267 of SEQ ID NO: 1 any of the following amino acids: Serine, Leucine, Threonine, Cysteine, Isoleucine, Valine or Alanine, preferably a Serine or Threonine; and/or b) to position 291 of SEQ ID NO: 1 an Isoleucine, Serine, Cysteine, Valine, Phenylalanine or Threonine, preferably a Threonine, Phenylalanine or Valine; or when the position corresponding to position 267 of SEQ ID NO: 1 is an Asparagine the position corresponding to position 291 of SEQ ID NO: 1 a Serine, Cysteine, Valine, Phenylalanine or Threonine, preferably a Threonine, Phenylalanine or Valine; In another aspect of the invention, in addition to the characteristics of the previous sentence the synthetic beta santalene synthase has the position corresponding to position 285 of SEQ ID NO: 1 filled with a Valine, the position corresponding to position 282 of SEQ ID NO: 1 filled with a Glutamine or Arginine, the position corresponding to position 271 of SEQ ID NO: 1 filled with a Serine, the position corresponding to position 273 of SEQ ID NO: 1 filled with a Alanine and/or the position corresponding to position 274 of SEQ ID NO: 1 filled with a Valine.

Moreover, the improved santalene synthases are those that carry in the position corresponding to position 291 of SEQ ID NO: 1 an Isoleucine, Valine, Methionine, Cysteine, Serine, Phenylalanine or Threonine, preferably Valine, Cysteine, Serine, Phenylalanine or Threonine, more preferably Cysteine, Threonine or Valine or alternatively for improved alpha santalene synthases a Leucine, and in addition the positions corresponding to positions in SEQ ID NO: 1 are filled with the amino acids listed for the corresponding position of SEQ ID NO: 1 in Table A′, B′ or C′ below.

TABLE A’

| Position in | |
|---|---|
| SEQ ID NO: 1_ | Amino acid(s) allowed |
| 267 | Asparagine, Serine, Leucine, Threonine, Cysteine, |
| | Isoleucine, Valine, Tryptophan, Glycine or an Alanine, |
| | preferably Asparagine, Serine, Threonine, Tryptophan, |
| | Glycine, Alanine or Leucine, more preferably Serine, |
| | Threonine, Glycine, Alanine or Leucine |
| 271 | Serine or Alanine, preferably Serine |
| 272 | Isoleucine, Valine, Methionine or Leucine, preferably |
| | Isoleucine |
| 273 | Alanine or Glycine, preferably Alanine |
| 274 | Valine or Methionine or Isoleucine or Leucine, preferably |
| | Valine |
| 275 | Valine or Methionine or Isoleucine or Leucine, preferably |
| | Valine |
| 276 | Valine or Methionine, preferably Valine |
| 277 | Glutamate |
| 278 | Proline |
| 279 | Glutamine or Asparagine or Lysine, preferably Glutamine |
| 280 | Tyrosine or Phenylalanine, preferably Tyrosine |
| 281 | Serine or Lysine, preferably Serine |
| 282 | Glutamine or Asparagine or Arginine or Lysine or Serine |
| | or Leucine or Threonine, preferably Glutamine or |
| | Arginine or Lysine |
| 283 | Cysteine or Alanine or Glycine, preferably Cysteine |
| 284 | Arginine |
| 285 | Valine or Methionine or Isoleucine or Leucine, preferably |
| | Valine |
| 286 | Alanine or Glycine or Valine, preferably Alanine |
| 287 | Isoleucine Valine, Methionine or Leucine, preferably |
| | Isoleucine |
| 288 | Threonine or Valine or Serine, preferably Threonine |
| 289 | Lysine |
| 290 | Alanine or Isoleucine, preferably Alanine |
| 292 | Valine, Glycine, Methionine or Isoleucine, preferably |
| | Valine |
| 293 | Leucine or Valine, preferably Leucine |
| 294 | Methionine, Isoleucine or Valine, preferably Methionine |
| 295 | Threonine or Serine, preferably Threonine |
| 296 | Alanine, Valine or Leucine, preferably Alanine |
| 297 | Methionine or Valine, preferably Methionine |
| 298 | Aspartate |
| 299 | Aspartate |
| 300 | Phenylalanine or Tyrosine or Alanine or Isoleucine or |
| | Glycine or Threonine, preferably Phenylalanine |
| 301 | Tyrosine or Phenylalanine, preferably Tyrosine |
| 302 | Aspartate |

TABLE B‘

| Position in | |
|---|---|
| SEQ ID NO: 1 | Amino acid(s) allowed |
| 267 | Asparagine, Serine, Threonine, Tryptophan, Glycine, |
| | Alanine or Leucine, preferably Serine, Threonine or Leucine |
| 271 | Serine |
| 272 | Isoleucine Valine, Methionine or Leucine, preferably |
| | Isoleucine |
| 273 | Alanine |
| 274 | Valine |
| 275 | Valine |
| 276 | Valine |
| 277 | Glutamate |
| 278 | Proline |
| 279 | Glutamine |
| 280 | Tyrosine |
| 281 | Serine |
| 282 | Glutamine or Arginine or Lysine |
| 283 | Cysteine |
| 284 | Arginine |
| 285 | Valine |
| 286 | Alanine |
| 287 | Isoleucine |
| 288 | Threonine |
| 289 | Lysine |
| 290 | Alanine |
| 292 | Valine |
| 293 | Leucine |
| 294 | Methionine |
| 295 | Threonine |
| 296 | Alanine |
| 297 | Methionine |
| 298 | Aspartate |
| 299 | Aspartate |
| 300 | Phenylalanine or Tyrosine or Alanine, or Glycine or |
| | Threonine, preferably Phenylalanine |
| 301 | Tyrosine or Phenylalanine, preferably Tyrosine |
| 302 | Aspartate |

TABLE C‘

| Position in | |
|---|---|
| SEQ ID | |
| NO: 1 | Amino acid(s) allowed |
| 267 | Asparagine, Serine, Threonine, |
| | Tryptophan, Glycine, Alanine |
| | or Leucine, preferably Serine, |
| | Threonine or Leucine |
| 271 | Serine |
| 272 | Isoleucine |
| 273 | Alanine |
| 274 | Valine |
| 275 | Valine |
| 276 | Valine |
| 277 | Glutamate |
| 278 | Proline |
| 279 | Glutamine |
| 280 | Tyrosine |
| 281 | Serine |
| 282 | Glutamine |
| 283 | Cysteine |
| 284 | Arginine |
| 285 | Valine |
| 286 | Alanine |
| 287 | Isoleucine |
| 288 | Threonine |
| 289 | Lysine |
| 290 | Alanine |
| 292 | Valine |
| 293 | Leucine |
| 294 | Methionine |
| 295 | Threonine |
| 296 | Alanine |
| 297 | Methionine |
| 298 | Aspartate |
| 299 | Aspartate |
| 300 | Phenylalanine |
| 301 | Tyrosine |
| 302 | Aspartate |

In an aspect of the invention the improved santalene synthases have at the position that corresponds to the position 267 of SEQ ID NO: 1 the amino acid found at position 267 of the polypeptide of any the following SEQ ID Nos: 2, 3 or 29, and at the position that corresponds to position 291 of SEQ ID NO: 1 the amino acid found at position 291 of the polypeptide of any the following SEQ ID Nos: 30, 31, 32, 33 or 34 for improved beta santalene synthases, or of SEQ ID NO: 53 for improved alpha santalene synthases, and have at least 50%, 60%, 65%, 70%, 75%, 80%, 85% 86% 87%, 88% 89% 90% 91%, 92% 93% 94% 95% 96% 97% 98%, 99% or 100% sequence identity over the full length to any of polypeptides of the SEQ ID NO: 2, 3, 29 to 40 or 53.

In a further aspect of the invention, the improved santalene synthases have the following amino acid residues listed in Table D at the positions corresponding to the positions in SEQ ID NO: 1 provided in Table D, and preferably the position corresponding to position 267 of SEQ ID NO: 1 is filled with a Serine, Threonine, Tryptophan, Glycine, Alanine or Leucine, preferably Serine, Threonine or Leucine, and more preferably the position corresponding to the position 255 of SEQ ID NO: 1 is filled with an amino acid with a hydrophobic side chain or a polar uncharged side chain, preferably Serine, Threonine, Alanine or Valine, more preferably Alanine.

TABLE D

| Position in | |
|---|---|
| SEQ ID | |
| NO: 1 | Amino acid(s) allowed |
| 271 | Serine or Alanine |
| 272 | Isoleucine |
| 273 | Alanine or Glycine |
| 274 | Valine or Methionine |
| 275 | Valine |
| 276 | Valine |
| 277 | Glutamate |
| 278 | Proline |
| 279 | Glutamine |
| 280 | Tyrosine |
| 281 | Serine |
| 282 | Glutamine or Leucine or |
| | Arginine or Lysine |
| 283 | Cysteine |
| 284 | Arginine |
| 285 | Valine or Isoleucine |
| 286 | Alanine or Valine |
| 287 | Isoleucine |
| 288 | Threonine |
| 289 | Lysine |
| 290 | Alanine |
| 291 | Valine, Cysteine, Serine, |
| | Leucine, Phenylalanine or |
| | Threonine, preferably Threonine, |
| | Serine, Phenylalanine or Valine, |
| | more preferably Threonine, |
| | Serine or Valine |
| 292 | Valine or Glycine |
| 293 | Leucine |
| 294 | Methionine or Isoleucine |
| 295 | Threonine |
| 296 | Alanine or Valine |
| 297 | Methionine |
| 298 | Aspartate |
| 299 | Aspartate |
| 300 | Phenylalanine or Isoleucine |
| 301 | Tyrosine |
| 302 | Aspartate |

In a further preferred aspect of the invention, the improved santalene synthases have in addition to the favourable amino acids at the positions corresponding to positions 267 and 291 of SEQ ID NO: 1, the following amino acids: a Serine (271), Alanine (273), Valine (274), Glutamine (282), Valine (285), Alanine (286), Valine (292), Methionine (294), Alanine (296) and Phenylalanine (300) at the positions that correspond to position of SEQ ID NO: 1 provided in brackets next to each amino acid listed here.

In another preferred aspect of the invention the improved santalene synthases have in addition to the favourable amino acids at the positions listed above an Arginine at the position corresponding to the position 232 in SEQ ID NO: 1.

Table 1 shows the ratios of beta-santalene to alpha-santalene in some of the improved santalene synthases and controls:

| | | | | | | | | | Wild- | |
|---|---|---|---|---|---|---|---|---|---|---|
| Designation: | I291V | I291T | I291S | I291C | I291F | N267S | N267G | N267A | type | I291L |
| SEQ ID NO: | 30 | 31 | 32 | 33 | 34 | 2 | 57 | 58 | 1 | 53 |
| Ratio b/a | 1.51 | 1.13 | 1.50 | 1.14 | 4.27 | 5.96 | 5.70 | 6.18 | 0.43 | 0.24 |

Designation: santalene synthase tested;
Ratio b/a: Ratio of beta-santalene to alpha-santalene w %/w %

Skillful improvements resulted in increased beta-santalene to alpha-santalene ratio or increased alpha santalene in the products of the improved santalene synthases as shown in table 1. Entries in italics are for the unmodified enzymes of SEQ ID NO: 1 (“wildtype”) and for I291 L, which produce an excess of alpha santalene. The later shows that skillful modification at the given positions will result in a desired improvement of either the beta-santalene to alpha-santalene ratio or the alpha-santalene production, as the I291 L modification allows to produce larger amounts of alpha-santalene than the unmodified enzyme of SEQ ID NO: 1, as shown by the lower beta-santalene to alpha-santalene ratio of I291L.

Increasing or decreasing the beta-santalene produced requires an inventive choice of the amino acid at positions 267 and/or 291. For example the inventors replaced Isoleucine at the position corresponding to position 291 of SEQ ID NO: 1 by Leucine (see SEQ ID NO: 53) to increase the alpha-santalene production over SEQ ID NO: 1, but at the expense that beta-santalene and bergamotene are not improved but rather decreased. One aspect of the invention is therefore to a synthetic alpha santalene synthase having a Leucine at the position that corresponds to the position 291 of SEQ ID NO: 1 with improved production of alpha-santalene compared to the unmodified enzyme.

When a Histidine was introduced at position 291 of SEQ ID NO: 1, the activity of the santalene synthase was destroyed and alpha-santalene, beta-santalene and bergamotene were not produced. In one aspect of the invention, Improved santalene synthases according to the invention have at the position 291 an amino acid other than Histidine.

In one aspect the improved santalene synthases of the invention do not have a Histidine or Glycine residue at the position that corresponds to positions 291 of SEQ ID NO: 1, but an Isoleucine, Valine, Threonine, Cysteine, Phenylalanine or Serine, preferably Cysteine, Valine, Serine, Phenylalanine or Threonine, or in case increased alpha santalene to beta santalene ratios are desired, a Leucine at the position corresponding to position 291 of SEQ IDNO: 1. In another aspect of the invention Isoleucine is found at the position that corresponds to positions 291 of SEQ ID NO: 1, when the position corresponding to position 267 of SEQ ID NO: 1 is filled with an Serine, Threonine or Leucine, or at that position 291 either a Valine, Cysteine, Serine, Phenylalanine or a Threonine is found when the position corresponding to position 267 of SEQ ID NO: 1 is filled with an Asparagine.

In another preferred embodiment the improved santalene synthases comprise a Arginine (261), Leucine (264), a Leucine (265), a Serine (271), an Alanine (273), a Proline (278), an Arginine (284), a Isoleucine (287), an Aspartate (298), an Aspartate (299) and an Aspartate (302) at the positions that correspond to position of SEQ ID NO: 1 provided in brackets next to each amino acid listed here, and preferably the position corresponding to position 267 of SEQ ID NO: 1 is filled with a Serine, Leucine, Threonine, Cysteine, Isoleucine, Valine, Tryptophan, Glycine or an Alanine, preferably Serine, Threonine, Tryptophan, Glycine, Alanine or Leucine, more preferably Serine or Leucine. For improved beta santalene synthases in a further embodiment this position is filled with Asparagine and the position corresponding to position 291 of SEQ ID NO: 1 is filled with a Valine, Cysteine, Serine, Phenylalanine or Threonine.

In a further preferred embodiment, the improved santalene synthase in addition has at least 50%, 55%, 60%, 65%, 70%, 75%, 80%, 85%, 90%, more preferably at least 91%, 92%, 93%, 94%, 95%, 96%, 97%, 98% or 99% and even more preferred 100% of all those amino acids that are marked in FIG. 1 by black background shading as being strongly conserved.

In another preferred embodiment, the improved santalene synthases comprises a sequence of SEQ ID NO: 1, a variant, derivative, orthologue, paralogue or homologue thereof, in which the amino acid at position 267 is replaced by Leucine, Serine or Threonine, and the amino acid at position 291 is replaced by Threonine, Serine, Cysteine, Phenylalanine or Valine, or in case increased alpha santalene amounts are desired to be produced by a Leucine.

In another preferred embodiment, the improved beta santalene synthases comprise a sequence of SEQ ID NO: 1, a variant, derivative, orthologue, paralogue or homologue thereof, in which the amino acid at position 267 is replaced by Leu, and the amino acid at position 291 is replaced by Thr.

In another preferred embodiment, the improved beta santalene synthases comprise a sequence of SEQ ID NO: 1, a variant, derivative, orthologue, paralogue or homologue thereof, in which the amino acid at position 267 is replaced by Leu, and the amino acid at position 291 is replaced by Ser.

In another preferred embodiment, the improved beta santalene synthases comprise a sequence of SEQ ID NO: 1, a variant, derivative, orthologue, paralogue or homologue thereof, in which the amino acid at position 267 is replaced by Leu, and the amino acid at position 291 is replaced by Cys or Phe.

In another preferred embodiment, the improved beta santalene synthases comprise a sequence of SEQ ID NO: 1, a variant, derivative, orthologue, paralogue or homologue thereof, in which the amino acid at position 267 is replaced by Leu, and the amino acid at position 291 is replaced by Val.

In another preferred embodiment, the improved beta santalene synthases comprise a sequence of SEQ ID NO: 1, a variant, derivative, orthologue, paralogue or homologue thereof, in which the amino acid at position 267 is replaced by Ser, and the amino acid at position 291 is replaced by Thr.

In another preferred embodiment, the improved beta santalene synthases comprise a sequence of SEQ ID NO: 1, a variant, derivative, orthologue, paralogue or homologue thereof, in which the amino acid at position 267 is replaced by Ser, and the amino acid at position 291 is replaced by Ser.

In another preferred embodiment, the improved beta santalene synthases comprise a sequence of SEQ ID NO: 1, a variant, derivative, orthologue, paralogue or homologue thereof, in which the amino acid at position 267 is replaced by Ser, and the amino acid at position 291 is replaced by Cys or Phe.

In another preferred embodiment, the improved beta santalene synthases comprise a sequence of SEQ ID NO: 1, a variant, derivative, orthologue, paralogue or homologue thereof, in which the amino acid at position 267 is replaced by Ser, and the amino acid at position 291 is replaced by Val.

The improved santalene synthases have typically of a molecular weight between 60 and 70 kDa, preferably between 61 and 66 kDa without any tags, added domains or fusions to other protein parts.

In a preferred embodiment, the improved santalene synthase has at least 50%, 55%, 60%, 65%, 70%, 75%, 80%, 85%, 90%, for example at least, 91%, 92%, 93%, 94%, 95%, 96%, 97%, 98% or 99% and for example 100% sequence identity over the full length of SEQI DNO: 1. In a further preferred embodiment, the improved santalene synthase has at least 50%, 55%, 60%, 65%, 70%, 75%, 80%, 85%, 90%, for example at least, 91%, 92%, 93%, 94%, 95%, 96%, 97%, 98% or 99% and for example 100% sequence identity over the full length of the protein sequence of any of SEQ ID NO: 2, 3, 14 to 17, 21 to 52, preferably any of SEQ ID NO: 2, 3, 29 to 40, for improved beta santalene synthases—or if increased alpha santalene production is desired at least 50%, 55%, 60%, 65%, 70%, 75%, 80%, 85%, 90%, for example at least, 91%, 92%, 93%, 94%, 95%, 96%, 97%, 98% or 99% and for example 100% sequence identity over the full length of protein sequence of SEQ ID NO: 13, 18, 19, 20 or 53, preferably of SEQ ID NO: 53—and more preferably has in addition all those amino acids that are marked in FIG. 1 by black background shading as being strongly conserved.

In santalene synthases with increased beta-santalene to alpha-santalene ratio, preferably a) the position corresponding to position 267 of SEQ ID NO: 1 is filled with a Serine, Leucine, Threonine, Cysteine, Isoleucine, Valine, Tryptophan, Glycine or an Alanine, preferably Serine, Threonine, Tryptophan, Glycine, Alanine or Leucine, more preferably Serine or Leucine, or the position corresponding to position 291 of SEQ ID NO:1 is filled with Valine, Threonine, Cysteine, Phenylalanine or Serine, more preferably with Thr, Val, Cys or Ser, or b) the position corresponding to position 267 in SEQ ID NO: 1 is an Asparagine and the position corresponding to position 291 of SEQ ID NO:1 is filled with Valine, Threonine, Cysteine, Phenylalanine or Serine, more preferably with Thr, Val, Cys or Ser; or c) the position corresponding to position 267 of SEQ ID NO: 1 is filled with a Serine, Leucine, Threonine, Cysteine, Isoleucine, Valine, Tryptophan, Glycine or an Alanine, preferably Serine, Threonine, Tryptophan, Glycine, Alanine or Leucine, more preferably Serine, Glycine, Alanine or Leucine and the position corresponding to position 291 of SEQ ID NO:1 is filled with an Isoleucine, Valine, Threonine or Methionine; or d) a combination of a), b) or c) with an Alanine residue at a position corresponding to position 255 of SEQ ID NO: 1; ore) a combination of a), b) c) or d) with a Histidine in the position that corresponds to position 268 of SEQ ID NO: 1.

In Santalene synthases with increased alpha-santalene to beta-santalene ratio, the position corresponding to position 291 of SEQ ID NO:1 is filled with Leucine, and the position corresponding to position 267 in SEQ ID NO: 1 is an Asparagine, Serine, Threonine or Leucine, preferably an Asparagine.

One aspect of the invention relates to synthetic santalene synthases producing alpha-santalene in excess of beta-santalene from farnesyl pyrophosphate, wherein the santalene synthase has at least 50%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to the amino acid positions 261 to 302 of any of SEQ ID NO: 1, 2, 3, 29, 57 or 58 using an Arginine residue that corresponds to the Arginine at position 261 of SEQ ID No: 1, 2, 3, 29, 57 or 58 and three Aspartate residues that correspond to the Aspartates at positions 298, 299 and 302 of SEQ ID NO: 1, 2, 3, 29, 57 or 58 to align the two protein sequences for the sequence identity determination and wherein the position corresponding to the position 291 in SEQ ID NO: 2 or 3 is a Glycine or Leucine, preferably Leucine. In one aspect these improved alpha santalene synthases have at the position corresponding to position 267 of SEQID NO: 1 an Asparagine.

Preferably, the improved beta santalene synthase of the invention has at least 50%, preferably at least 60%, at least 70%, at least 80%, at least 90% or at least 95% sequence identity to any of SEQ ID NO: 2, 3, 14 to 17, 21 to 52, 57 or 58, preferably any of SEQ IDNO: 2, 3, 29 to 40, 57 or 58 in the part of the protein that starts with an Arginine in the position that corresponds to the Arginine in position 261 of SEQ ID NO: 2, 3, 29, 57 or 58 and stretches to three Aspartates in positions corresponding to the Aspartates at positions 298, 299 and 302 of SEQ ID NO: 2, 3 or 29 to 40, 57 or 58 and has at the position corresponding to position 267 of SEQ ID NO: 2, 3, 29, 57 or 58 a Serine, Leucine, Threonine, Cysteine, Isoleucine, Valine, Tryptophan, Glycine or Alanine, preferably a Serine, Threonine, Tryptophan, Glycine, Alanine or Leucine, and/or at the position corresponding to position 291 of SEQ ID NO: 2, 3, 29, 57 or 58 a Valine, Cysteine, Serine, Phenylalanine or Threonine, preferably Valine, Serine, Phenylalanine or Threonine, or in case at the position corresponding to position 267 of SEQ ID NO: 2, 3, 29, 57 or 58 a Serine, Threonine, Tryptophan, Glycine, Alanine or Leucine is present, an Isoleucine at the position corresponding to the position 291 of SEQ ID NO: 2, 3 or 29.

In another preferred aspect, the improved beta santalene synthase has at least 50%, preferably at least 60%, at least 70% or at least 80% sequence identity to SEQ ID NO: 2, 3, 14 to 17, 21 to 52, 57 or 58, preferably any of SEQ IDNO: 2, 3 or 29 to 40, 57 or 58 in the part of the protein that starts with an Arginine in the position that corresponds to the Arginine in position 261 of SEQ ID NO: 2, 3, 29, 57 or 58 and stretches to three Aspartates in positions corresponding to the Aspartates at positions 298, 299 and 302 of SEQ ID NO: 2, 3, 29, 57 or 58, and has at the position corresponding to position 267 of SEQ ID NO: 2, 3, 29, 57 or 58 an Asparagine, Serine, Leucine, Threonine, Cysteine, Isoleucine, Valine, Tryptophan, Glycine or Alanine, preferably an Asparagine, Serine, Threonine, Tryptophan, Glycine, Alanine or Leucine, and/or at the position corresponding to position 291 of SEQ ID NO: 2, 3, 29, 57 or 58 a Valine, Serine, Cysteine, Phenylalanine or Threonine, preferably Serine, Valine, Phenylalanine or Threonine and preferably has a Histidine at the position that corresponds to position 268 in SEQ ID NO: 2, 3, 29, 57 or 58.

The amounts of beta-santalene and alpha-santalene are determined by a reliable quantitative method, preferably gas chromatography with a FID detector. A preferred method for determining the amounts of alpha-santalene, beta-santalene and bergamotene is described in detail in the examples section.

The improved beta santalene synthases produce beta-santalene in excess of alpha-santalene which means under conditions suitable for the production of these santalenes, the enzymes produce beta-santalene and alpha-santalene in a molar ratio of beta-santalene to alpha-santalene that is greater than 1.0. The improved alpha santalene synthases produce alpha-santalene in excess of beta-santalene which means under conditions suitable for the production of these santalenes, the enzymes produce beta-santalene and alpha-santalene in a molar ratio of beta-santalene to alpha-santalene that is lower than 1.0.

Suitable conditions for the production of these santalenes can for example be provided by expression of the DNA encoding for the improved santalene synthase in a host cell that provides for active improved santalene synthases and provides for all substrates and co-factors e.g. farnesylpyrophosphate and Magnesium ions, for the improved enzyme to perform the reactions to the alpha- and beta-santalene.

So far, known santalene synthases produce a composition in which the molar ratio of beta-santalene to alpha-santalene is below 1. The improved beta santalene synthases of the invention produce beta-santalene and alpha-santalene, preferably measured by GC-FID, in a molar ratio of beta-santalene to alpha-santalene that is equal to or greater than 1; for example the ratio is at least 1.05, 1.1, 1.2, 1.3, 1.4, 1.5, 1.6, 1.7, 1.8, 1.9 or at least 2. The ratio of beta-santalene to alpha-santalene may be at least 3:1, preferably at least 4:1, more preferably at least 5:1, even more preferably 6:1, yet even more preferably at least 7:1, most preferably at least 8:1 and even at least 9:1. In one aspect of the invention, the ratio is not greater than 100:1.

One aspect of the invention relates to a synthetic nucleic acid encoding for any of the synthetic santalene synthases of the invention, either the santalene synthases with increased beta-santalene to alpha santalene production (for example but not limited to the polypeptides of SEQ ID NO: SEQ ID NO: 2, 3, 14 to 17, 21 to 52, or variants thereof), or the ones with improved alpha santalene production compared to the natural santalene synthases before modification for example but not limited to the polypeptide of SEQ ID NO: 53 or variants thereof. A further part of the inventions is an expression cassette comprising the synthetic nucleic acid of the invention.

A further preferred embodiment is a method for producing a composition with a surplus of beta-santalene over alpha-santalene, preferably a method suitable for large scale production, using the improved beta santalene synthases disclosed herein, including the steps of i) providing one or more improved beta santalene synthase in an active form and with all required co-factors for example but not limited to metal ions like magnesium ions, ii) contacting farnesyl pyrophosphate with the one or more improved beta santalene synthases under conditions permitting the production of santalenes, iii) producing beta-santalene and alpha santalene and optionally bergamotene and optionally other santalenes from farnesyl pyrophosphate, wherein the amount of beta-santalene produced is larger than the amount of alpha-santalene produced and optionally purification of the products for example to separate them from the santalene synthases and any remaining substrate and undesired compounds. Preferably, these methods produce compositions that comprise more beta-santalene than alpha santalene in a molar ratio of the two that is at least 1.1, 1.2, 1.3, 1.4, 1.5, 1.6, 1.7, 1.8, 1.9 or at least 2; the ratio of beta-santalene to alpha-santalene may be at least 3:1, preferably at least 4:1, more preferably at least 5:1, even more preferably 6:1, yet even more preferably at least 7:1, most preferably at least 8:1 and even at least 9:1. In one aspect of the invention, the ratio is not greater than 100:1.

It will be particularly beneficial to perform the method for producing a composition with a surplus of beta-santalene over alpha-santalene including fermentation steps to provide the improved beta santalene synthase and contacting farnesyl pyrophosphate with it and producing the santalenes. Although for example, methods using isolated santalene synthases of the invention in vitro are possible, particularly preferred therefore is a fermentative method for the production of a composition comprising beta-santalene in excess of alpha-santalene comprising the following steps:
a) Providing a nucleic acid encoding the improved beta santalene synthase in a manner suitable to be expressed in a host,
b) Introducing the nucleic acid of a) into a host cell that is able to provide farnesyl pyrophosphate and all necessary co-factors to the santalene synthase to be active,
c) Cultivating the host cell in a manner that it produces the santalene synthase encoded by the nucleic acid of a) in an active form and provides farnesyl pyrophosphate and all necessary co-factors to the santalene synthase,
d) Producing beta-santalene and alpha-santalene and optionally bergamotene from farnesyl pyrophosphate with the help of the improved beta santalene synthase, wherein the amount of beta-santalene produced is larger than the amount of alpha-santalene produced,
e) Harvesting the produced beta-santalene and alpha-santalene and optionally bergamotene when the desired amounts of these compounds have been produced,
f) Optionally purifying the beta-santalene and alpha-santalene and optional bergamotene.

The amount of beta-santalene produced by the improved beta santalene synthases and by the methods of the invention comprise on a weight per weight basis in increasing order of preference at least 10%, 20%, 30%, 40%, 50%, 60%, 70%, 80%, 90% or 95% more beta-santalene compared to those of produced by the unmodified santalene synthase under identical conditions. Optionally, the amount of bergamotene produced by the improved beta santalene synthases and by the methods of the invention comprise on a weight per weight basis in increasing order of preference at least 10%, 20%, 30%, 40%, 50%, 60%, 70%, 80%, 90% or 95% more bergamotene compared to those of produced by the unmodified santalene synthase under identical conditions. In one aspect of the invention, at least 12% (w/w), 18% (w/w) or 20% (w/w) bergamotene are produced by the improved santalene synthases and the methods of the invention. Even more preferably, at least twice the amount of beta-santalene and optionally bergamotene is present in the compositions produced.

In one aspect of the invention, the invention further relates to santalene compositions produced with the help of the improved beta santalene synthases that have a greater beta-santalene content than alpha-santalene content. In one aspect of the invention, inventive compositions are produced by one or more synthetic beta santalene synthase, the method(s) or the host cell(s) of the invention, wherein the composition comprises beta-santalene in excess to alpha-santalene. One preferred embodiment is a composition comprising, preferably substantially consisting of beta-santalene and alpha-santalene and bergamotene that is produced with the help of the improved beta santalene synthases, wherein the composition has beta-santalene in excess of alpha-santalene. In a particular aspect of the invention, the composition comprises more beta-santalene than bergamotene, and more bergamotene than alpha-santalene Inventive compositions preferably comprise more beta-santalene than alpha santalene in a ratio of the two that is greater than 1, for example the ratio is at least 1.1, 1.2, 1.3, 1.4, 1.5, 1.6, 1.7, 1.8, 1.9 or at least 2. The ratio of beta-santalene to alpha-santalene may be at least 3:1, preferably at least 4:1, more preferably at least 5:1, even more preferably 6:1, yet even more preferably at least 7:1, most preferably at least 8:1 and even at least 9:1. In one aspect of the invention, the ratio is not greater than 1000:1.

The invention further relates to compositions produced with the help of the improved beta santalene synthases that have a greater bergamotene content than alpha-santalene content. Such compositions comprise more bergamotene than alpha santalene in a ratio of the two that is greater than 1, preferably the ratio is at least 1.1, 1.2, 1.3, 1.4, 1.5, 1.6, 1.7, 1.8, 1.9 or at least 2. The ratio of bergamotene to alpha-santalene may be at least 3:1, preferably at least 4:1, more preferably at least 5:1, even more preferably 6:1, yet even more preferably at least 7:1, most preferably at least 8:1 and even at least 9:1. In one aspect of the invention, the ratio is not greater than 1000:1.

In one aspect of the invention, the compositions produced In one aspect of the invention, with the help of the improved santalene synthases comprise at least 12% (w/w), 18% (w/w) or 20% (w/w) bergamotene.

The ratio of bergamotene to beta-santalene produced by the improved santalene synthases and found in the compositions of the invention can be above 1 (bergamotene excess) or below 1 (beta-santalene excess). The first is the case for the compositions for example produced with the help of N267S (SEQ ID NO: 2) or the alpha santalene overproducer I291 L (SEQ ID NO: 53), the latter is exemplified by the compositions produced with the help of N267L (Seq ID NO.3), or any of SEQ ID NO: 30 to 34 or 36 or 37, as can be seen in FIGS. 4 and 5. Depending on the desired product and further processing of the composition, it is advantageous to use an improved beta santalene synthase of the bergamotene excess type, or one of the beta-santalene excess type. The improved beta-santalene synthases N267G (SEQ ID NO: 57) and N267A (SEQ ID NO: 58) showed an amount of bergamotene that was nearly at the level of beta-santalene, with strongly reduced alpha-santalene production, which also may be desirable for some uses.

In one aspect of the invention, the ratio of bergamotene to beta-santalene is below 1.0, for example equal to or below 0.95, 0.9, 0.85, 0.8 or 0.75, for example equal to or below 0.70, but higher than 0.28, for example higher than 0.30.

In one embodiment, the ratio of bergamotene to beta-santalene in the compositions produced with the help of the improved beta santalene synthase is at least 1:1. In one aspect of the invention, the ratio is not higher than 5.5:1, for example not higher than 5:1 or 4.5 to 1, or 4:1, or 3.5 to 1 or 3 to 1, or 2.5 to 1, or 2:1.

In another embodiment, the ratio of bergamotene to beta-santalene in the compositions produced with the help of the improved beta santalene synthase is 1:2, 1:3, 1:4, 1:5 or 1:10 or less. Therefore, in one aspect the invention relates to the improved beta santalene synthase, host cells of the invention or the methods of the inventions wherein the santalene synthase produces an excess of trans-α-bergamotene over alpha-santalene in addition to producing more beta-santalene than alpha-santalene.

A further embodiment is directed to a composition comprising more bergamotene than beta-santalene, and more beta-santalene than alpha-santalene producible by the improved beta santalene synthase, host cells of the invention or the methods of the inventions. Preferably, the compositions are produced including fermentative steps for either the production of the improved beta santalene synthases, or for the production of the composition.

In a preferred embodiment, the composition with more beta-santalene than alpha-santalene is obtained by cultivation of one or more types of host cells, preferably bacteria, plant or fungal (including yeast) cells, more preferably bacteria, even more preferably Escherichia coli, Amycolatopsis sp or Rhodobacter sphaeroides.

In a further preferred embodiment the invention relates compositions comprising β-santalol ((2Z)-2-Methyl-5-[2-methyl-3-methylene-bicyclo[2.2.1]hept-2-yl]pent-2-en-1-ol; CAS number 77-42-9) and α-santalol ((Z)-5-(2,3-Dimethyltricyclol[2.2.1.0^{2,6}]hept-3-yl)-2-methylpent-2-en-1-ol, CAS number 115-71-9) produced from a precursor composition comprising both beta-santalene and alpha santalene produced by the methods of the invention, wherein the β-santalol (also called beta-santalol herein) is present in greater amounts on a w/w basis than the α-santalol (also called alpha santalol herein) due to a surplus beta santalene content in the precursor composition. The beta-santalol to alpha santalol ratio in these compositions is greater than 1, preferably the ratio is at least 1.1, 1.2, 1.3, 1.4, 1.5, 1.6, 1.7, 1.8, 1.9 or at least 2. The ratio of beta-santalol to alpha-santalol may be at least 3:1, preferably at least 4:1, more preferably at least 5:1, even more preferably 6:1, yet even more preferably at least 7:1, most preferably at least 8:1 and even at least 9:1. In one aspect of the invention, the ratio is not greater than 100:1.

A further preferred embodiment is a method for producing a composition with a surplus of β-santalol over α-santalol without the need to a) diminish the alpha-santalene content before the conversion to alpha-santalol and/or b) to increase the beta-santalol content after the conversion from santalenes by distillation or other means, wherein the method comprises the steps of producing a composition with a surplus of beta-santalene over alpha-santalene by the methods of the invention, and in one or more subsequent steps oxidising the beta-santalene to β-santalol and the alpha-santalene to α-santalol. This conversion of the santalenes may be done biosynthetically and/or chemically to their respective alcohols. Following the conversion to santalols, purification steps like a distillation to remove other compounds may be included, and if desired the ratio of beta-santalol to alpha-santalol may be altered by distillation, but a composition with more beta-santalol than alpha-santalol can be achieved without further alterations of the beta-santalol to alpha-santalol ratio following the provision of the composition with beta-santalene in excess of alpha-santalene by the use of the improved beta santalene synthases. One aspect of the invention hence is a method for the production of a composition comprising beta-santalol in excess to alpha-santalol, wherein the method comprises the steps of producing a composition with a surplus of beta-santalene over alpha-santalene by the methods of the invention, and in one or more subsequent steps oxidising the beta-santalene to β-santalol and the alpha-santalene to α-santalol and wherein a distillation of santalols following the oxidation of santalenes is performed for purification of the santalols without increasing the beta-santalol content over the alpha-santalol content substantially.

Also the invention relates to compositions comprising beta-santalol in excess to alpha-santalol produced by any of the methods of the invention, with the improved beta santalene synthases of the invention or with the host cells of the invention, optionally with the sum of bergamotols in the compositions being less than 10% (w/w) or even less than 8% (w/w). In another aspect, the inventive compositions comprising beta-santalol in excess to alpha-santalol produced by any of the methods of the invention, with the improved beta santalene synthases of the invention or with the host cells of the invention comprise less than 3% epi-β-santalol

One aspect of the invention relates to a synthetic santalene synthase producing beta-santalene in excess of alpha-santalene, a nucleic acid encoding such, an expression cassette comprising such nucleic acids, host cells comprising such expression cassettes, methods of the invention and compositions produced with the inventive enzymes and methods comprising beta-santalene and alpha-santalene and/or beta-santalol and alpha santalol with a ratio of beta-santalene to alpha-santalene or the ratio of beta-santalol to alpha-santalol, respectively, of at least equal to or greater than 1.3, 1.5 or 2.

Preferably the compositions of the invention are lipophilic compositions.

The beta-santalene, alpha santalene or bergamotene produced by the inventive methods or the compositions of the invention may be used in flavour or fragrance applications, in cosmetic uses, as insect repellent or insect attractant, or in agriculture e.g. for crop protection or animal raising.

One aspect of the invention is a host cell suitable to produce one or more improved santalene synthase from one or more nucleic acid encoding said improved santalene synthase(s) and suitable to provide the improved santalene synthase(s) with farnesyl pyrophosphate and all co-factors required for its activity wherein the host cell comprises such nucleic acid(s). A further preferred embodiment therefore is to host cells comprising the improved santalene synthases of the invention. A microorganism capable of producing the composition with more beta-santalene than alpha-santalene may be a fungal cell (including yeast) or a bacterium or a plant cell or an animal cell, for example from the group consisting of the genera Escherichia, Klebsiella, Helicobacter, Bacillus, Lactobacillus, Streptococcus, Amycolatopsis, Rhodobacter, Lactococcus, Pichia, Saccharomyces and Kluyveromyces. In a preferred embodiment, the one or more host cell suitable for the production of a composition with more beta-santalene than alpha-santalene is a bacterial cell selected from a) the group of Gram negative bacteria, such as Rhodobacter (e.g. R. sphaeroides, R.capsulatus), Agrobacterium, Paracoccus (e.g. P. carotinifaciens, P. zeaxanthinifaciens), or Escherichia; b) a bacterial cell selected from the group of Gram positive bacteria, such as Bacillus, Corynebacterium, Brevibacterium, Amycolatopis; c) a fungal cell selected from the group of Aspergillus, Blakeslea, Peniciliium, Phaffia (Xanthophyllomyces), Pichia, Saccharamoyces, Kluyveromyces, Yarrowia, and Hansenula; or d) a transgenic plant or culture comprising trans-genic plant cells, wherein the ocell is of a transgenic plant selected from Nicotiana spp, Cichorum intybus, lacuca sativa, Mentha spp, Artemisia annua, tuber forming plants, oil crops and trees; e) or a transgenic mushroom or culture comprising transgenic mushroom cells, wherein the microorganism is selected from Schizophyllum, Agaricus and Pleurotisi. More preferred organisms are microorganism belonging to the genus Escherichia, Saccharomyces, Pichia, Amycolatopsis, Rhodobacter or Paracoccus, and even more preferred those of the species E. coli, S.cerevisae, Rhodobacter sphaeroides or Amycolatopis sp.

A further embodiment is an expression cassette comprising the synthetic nucleic acid encoding the improved santalene synthases. These nucleic acids may be the ones listed as SEQ ID NO: 11 or 12, or those encoding the polypeptides of any of SEQ ID NO: SEQ ID NO: 2, 3, 14 to 17, 21 to 52, or for increased alpha santalene production the nucleic acids encoding the polypeptide of SEQ ID NO: 53. Other nucleic acids suitable in the expression cassettes for altered santalene production in a host cell are those encoding an improved santalene synthase such as but not limited to those disclosed in any of SEQ ID NO: 2, 3, 13 to 53. For a nucleic acid encoding a santalene synthase with increased alpha-santalene production the nucleic acid encoding the polypeptide of SEQ ID NO: 53 can be used in such expression cassettes and host cells. Said expression cassette may be contained in a vector, the nucleus, a plasmid an artificial chromosome or any other means that allows for the expression in the host cell in the desired strength and manner.

A further aspect of the invention is a method to purposefully alter the product profile of a santalene synthase by altering the flexibility of the tertiary structure that corresponds to Helix C of SEQ ID NO:1 and to Helix D of SEQ ID NO: land to the polypeptide chain linking the two in SEQ ID NO: 1. For example this method involves the step of changing the nucleic acid encoding the santalene synthase so that the amino acid at a position that corresponds to the position 267 of SEQ ID NO: 1 is a Serine, Leucine, Threonine, Cysteine, Isoleucine, Valine, Tryptophan, Glycine or an Alanine, preferably Serine, Threonine, Tryptophan, Glycine, Alanine or Leucine, for example Serine or Leucine and/or the step of altering the codon of a nucleic acid encoding a santalene synthase in a way that the codon corresponding to the codon for position 291 of SEQ ID NO: 1 now encodes a Leucine, Valine, Threonine, Cysteine or Serine, for example Thr, Val, Cys, Phe or Ser; followed by the steps of expressing the modified nucleic acid in a host cell suitable for the expression of the synthetic santalene synthases of the invention.

### DESCRIPTION OF FIGURES

FIG. 1 shows an alignment of the known santalene synthases CiCaSSy wildtype (SEQ ID NO: 1), SaSSY, SaSSy14, SspiSSy, SauSSy, ClaSSy and SaSSy134 (SEQ ID NO: 4 to 9, respectively). SaSSY134 is labelled with SEQ 280 in this alignment. Also included are the two improved beta santalene synthases N267S and N267L mutants (SEQ ID NO: 2 and 3) The alignment was done with the clustalw software using typical settings. Black background shading marks a strongly conserved residue, grey background shading a residue that is conserved in at least 50% of the aligned sequences, white shading marks non-conserved amino acids.

FIG. 2 shows a 3D model of CiCaSSy SEQ ID NO: 1 created with the PyrMol software. The Alpha helices are shown, and black marks the two helices Helix C (short black helix) and Helix D (longer black helix) of CiCaSSy.

FIG. 3 shows a graphical representation of the interaction of Helix C and Helix D in the wildtype CiCaSSy (A) and the N267S mutant (B). The alpha helix in the center of the images represents Helix D, the alpha helix to the left represents Helix C. The side chains of the two amino acids in position 267 are marked by dark grey

FIG. 4 shows the changes in the three main products alpha-Santalene, beta-santalene and bergamotene by improving the santalene synthase of SEQ ID NO: 1 (“Wildtype”). Values have been normalised on these three major products; minor products are not shown. Filled black bars represent alpha-santalene, empty bars represent bergamotene and diagonally lined bars represent beta-santalene. Replacing position 267 with a Serine as in SEQ ID NO: 2 (“N2675”) or a Leucine residue as in SEQ ID NO: 3 (“N267L”) allows the enzyme to produce more beta-santalene and more bergamotene (N267S) than alpha-santalene, or more bergamotene and still considerable alpha-santalene, but less beta-santalene (N267L). Data for two santalene synthases known in the art are shown for comparison (termed “SaSSy” and “SaSSY-134” in FIG. 4). The data was taken from the reported values in the art, see WO2015153501. The known santalene synthases (Wildtype ClCassy, SaSSY and SaSSy-134) show a larger production of alpha-santalene than of the other two compounds. The improved version of N267S and N267L show how this product profile can be altered according to the desired prevalence of either beta-santalene alone over alpha-santalene as by N267L or of both beta-santalene and bergamotene over alpha-santalene as by N267S.

FIG. 5 shows the changes in the three products alpha-Santalene, bergamotene and beta-santalene by improving the santalene synthase at the position that corresponds to position 291 of SEQ ID NO: 1 alone or in combination with modifications of the position that corresponds to position 267 of SEQ IDNO: 1. Minor products are not shown for improved clarity. Filled black bars represent alpha-santalene, empty bars represent bergamotene and diagonally lined bars represent beta-santalene. Wildtype (SEQ ID NO: 1) and the modified enzyme “I291L” are shown as controls. Replacing position 291 with a Leucine residue as in SEQ ID NO: 53 (“I291L”) did not change the fact that a surplus of alpha-santalene compared to beta-santalene and bergamotene is produced, on the contrary this modification enhances the production of alpha-santalol over the one of the wildtype enzyme as can be seen from the figure.

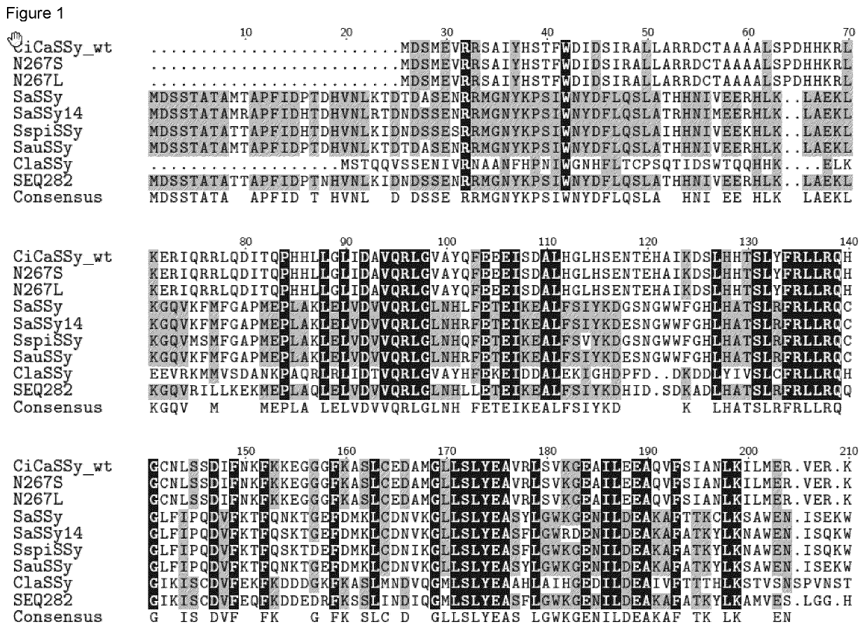

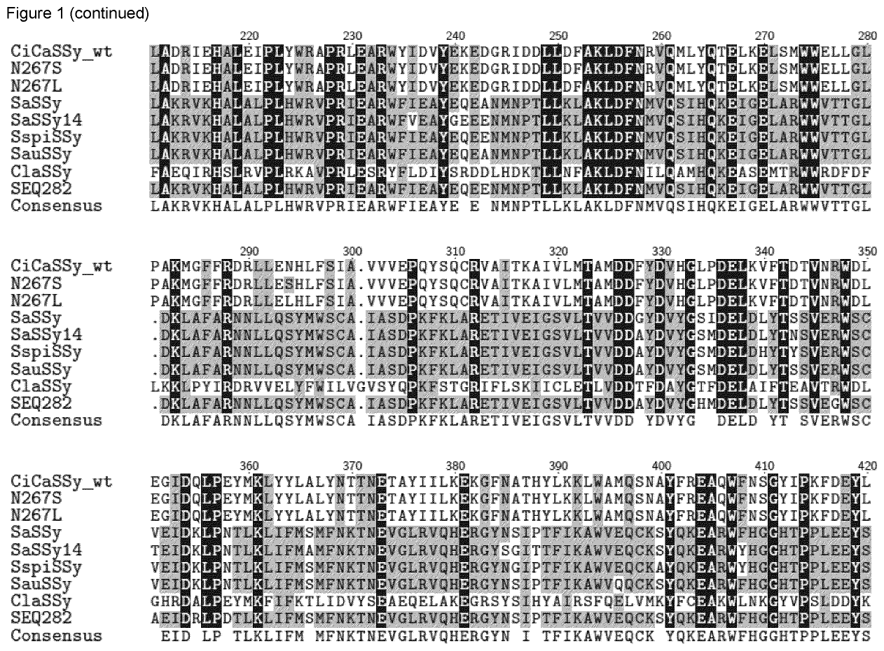

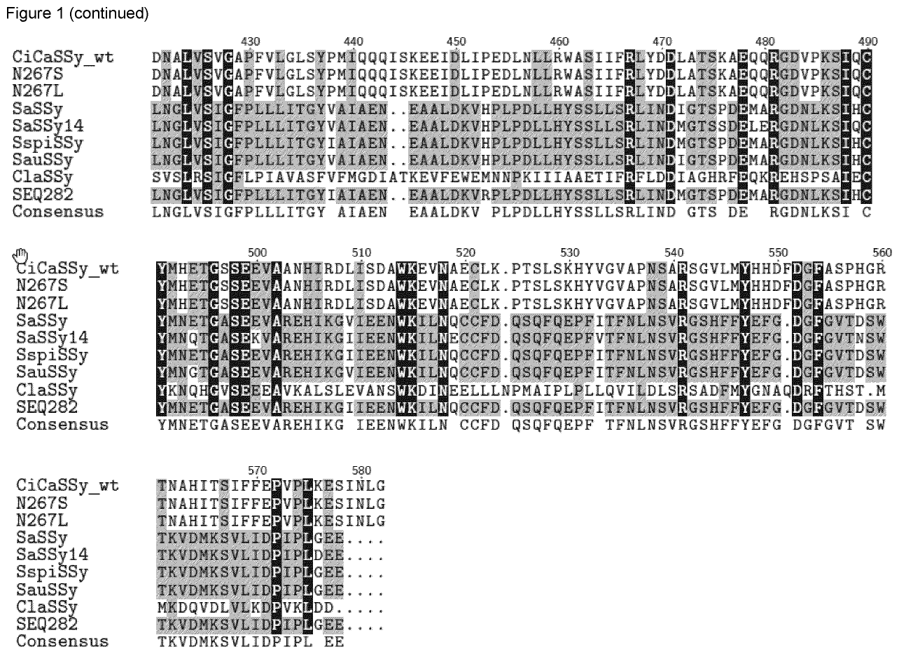

Replacing position 291 with a Valine, Serine, Threonine or Cysteine (“I291V”, “I291S”, “I291T” and “I291C”, respectively), allows the enzyme to produce more beta-santalene than alpha-santalene, yet maintain much larger levels of alpha-santalene than in the N267S version of the improved beta santalene synthase. The improved version of I291V, I291S, I291C and I291T show how this product profile can be altered according to the desired prevalence of either beta-santalene alone over alpha-santalene as by I291T, I291S and I291C, or of both beta-santalene over alpha-santalene and bergamotene at levels similar or above those of alpha-santalene as by I291V, yet maintaining larger alpha-santalene levels compared to the N267S improvement. Such a profile with more remaining alpha-santalene can be advantageous for some applications.

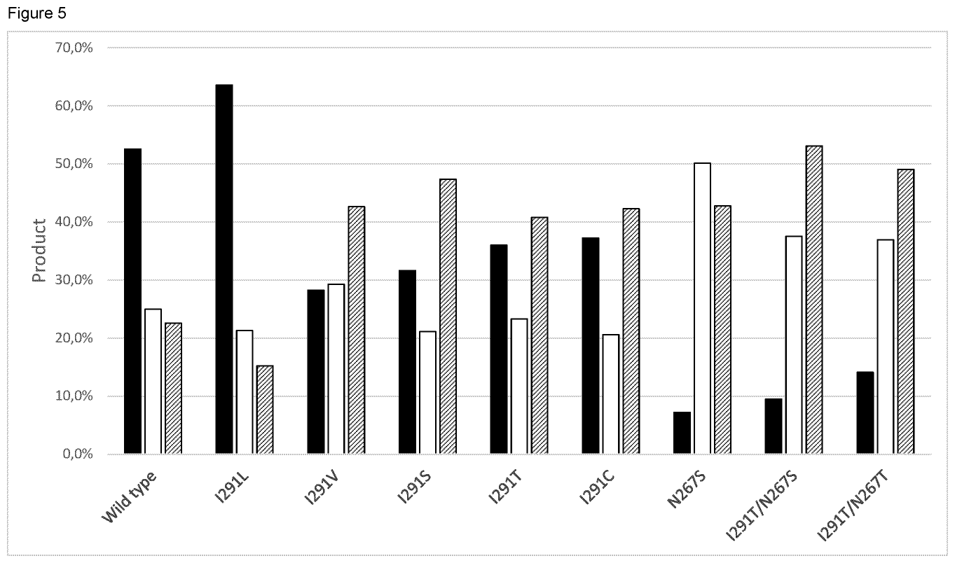

The last two groups of bars show the results for the two double mutants with the positions corresponding to positions 267 and 291 of SEQ ID NO: 1 being modified. The data shown for “I291T/N267S” is from an enzyme in which the position 267 was filled with a Serine, and the position 291 with a Threonine. The data shown for “I291T/N267T” is for one that had a Threonine introduced in both these positions. As can be seen, from the mutants shown in FIG. 5 the largest percentage of beta-santalene is produced by the double mutant “I291T/N267S”. The amounts of alpha-santalene and bergamotene for the “I291T/N267S” enzyme are a type of intermediate of these values for the two single mutants, with the mutation N267S having the more impact on these values than I291T in this combination. The data for the other double mutant shows that a Threonine at position 267 has similar effects on alpha-santalene, bergamotene and beta-santalene than a Serine at this position causes, yet not quite as strong.

### EXAMPLES

Publicly available electronic sequence information was used to analyse santalene synthase structures using standard software tools. A 3D model was generated of CiCaSSy (SEQ ID NO: 1), a santalene synthase from Cinnamomum camphora disclosed as SEQ ID NO 3 in the international patent application published as WO2018160066 with a normal alpha-santalene to beta-santalene ratio Common tools for such analysis are for example Structural alignment software: DALI, CE, STAMP; see http://www.rcsb.org/pdb/home/home.do for a choice.

The enzyme known as CiCaSSy has a bit unusual amino acid positioning compared to other santalene synthases. For example, it shares less than 50% sequence identity with many other santalene synthases yet combines elements from many other santalene synthases in some stretches. The active site cavity was identified, and residues within this were targeted for mutagenesis. In particular, residues that might influence the product profile were prioritized. An area comprising two spatially close α-helices in the middle of the amino acid sequence was chosen for mutations. CiCaSSy has in this area of the protein some difference in amino acids compared to each santalene synthases that are known, yet many elements at the same time are shared with different groups of santalene synthases in a combination only found in CiCaSSy. If this area of the protein is the key part for the product profile changes desired, transfer to other santalene sequences is easily feasible even if they differ in the remaining part to a great extent.

### Mutation Testing

After in depth study the residue 267 of CiCaSSy was chosen for mutation. The inventors realized that the surrounding of N267 in SEQ ID NO: 1 is so favourable that the inventors chose to replace that unusual asparagine at position 267 also with Serine and Leucine, although these are found at the corresponding location in other santalene synthases of known lacking performance. DNA sequences encoding CiCaSSy proteins with the two desired mutations at position 267 were synthesized. The resulting protein sequence named N267S and N267L are given in SEQ ID NO:2 and SEQ ID NO: 3, respectively.

A root-mean-square deviation of atomic positions (RMSD) and a root mean square fluctuation (RSMF) analysis was performed with these two novel protein sequences. Each enzyme was simulated for 500 ns in the same condition (pH 8.0, 300 K, 1 atm, water environment, ions present without substrate). RMSD provides an indication of the movements and flexibility of the overall protein while RSMF indicates the average movement and flexibility at a given position, The RSMF showed that the N267S showed the predicted increase in fluctuation and hence flexibility in the area of the loop between Helix C and Helix D and the part of Helix D that interacts with the side chain of position 267 in SEQ ID NO 1 to 3 over the wildtype CiCaSSy. It was observed that the flexibility of the stretch that corresponds to positions 272 to 291 (which is the area where the side chains of Helix D are located that will interact with the side chain of the amino acid at position 267) that was increased in N267S compared to the flexibility in the wildtype CiCaSSy. The increase was of higher magnitude in the stretch from 272 to 284 which contains the loop between Helix C and Helix D, which is expected to be less rigid than a helix of course. Both N267S and N267L had further stretches of increased fluctuations as indication of flexibility further downstream, in the area of positions 380 to 500. When this was compared with the RSMF analysis of other santalene synthases with alpha-santalene surplus production, this pattern was not observed in any of the sequences of SEQ ID NO: 4, 5, 8 or 9 analysed. RSMD analysis showed that for N267S after 30000 picoseconds the deviations in nm increased by about one fifth from the initial equilibrium. This flexibility in structure was not observed in any of the other santalene sequences analysed.

The procedure described for the wildtype CiCaSSy in examples 6 to 19 of WO2018160066 (p. 44, I.19 to p.50, I. 22; incorporated herein by reference) was applied for the experiments with the mutated CiCaSSy sequences encoding the proteins N267S and N267L. The mutated DNA sequence encoding the CiCaSSy santalene synthase of SEQ ID NO: 2 and 3 were introduced into Rhodobacter sphaeroides by the procedure disclosed in international patent application published as WO2018160066 for CiCaSSy (SEQ ID NO: 1 of the present invention), SEQ ID NO: 3 in WO2018160066 using a plasmid based system to express heterologously the DNA sequence and form the mutate enzyme. Fermentation of Rhodobacter sphaeroides for the production of, extraction of and analysis of alpha-santalene, beta-santalene and bergamotene produced by the host cells were performed as in WO2018160066.

The determination of alpha-santalene, beta-santalene and bergamotene was performed with gas chromatography with FID detector:

Gas chromatography was performed on a Shimadzu GC2010 Plus equipped with a Restek RTX-SSil MS capillary column (30 m×0.25 mm, 0.5 pm). The injector and FID detector temperatures were set to 280° C. and 300° C., respectively. Gas flow through the column was set at 40 mL/min. The oven initial temperature was 160° C. increased to 180° C. at a rate of 2° C./min, further increased to 300° C. at a rate of 50° C./min, and held at that temperature for 3 min. Injected sample volume was 1 μL with a 1:50 split-ratio, and the nitrogen makeup flow was 30 ml/min

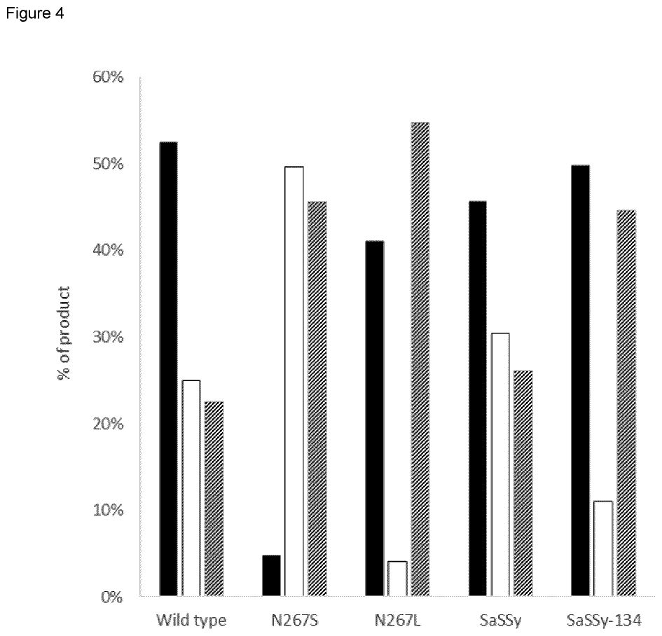

The two enzyme mutants at the 267 position of CiCaSSy; N267S and N267L, had a significant effect on the product ratios of alpha-santalene, beta-santalene and bergamotene. Both mutations led to an increased beta-santalene production compared to wildtype CiCaSSy, even producing more beta-santalene than alpha-santalene for the first time, and an increased beta-santalene to alpha-santalene product ratio (FIG. 4). Surprisingly, the N267S mutant also produced significantly less alpha-santalene, meaning this mutant had high specificity for beta-santalene over alpha-santalene—the first time such a phenomenon has been observed. The N267L mutation had an even larger change in product ratios, and produced beta-santalene as its major product, whilst producing relatively less alpha-santalene and trans-α bergamotene as shown in FIG. 4.

Additional mutants were tested with the same experimental set-up described above. For example, replacing the position corresponding to SEQ ID NO: 267 with a Glycine, Alanine or Tryptophan also resulted in improved santalene synthases.

Further, it was found that replacing the Isoleucine at the position corresponding to the position 291 of SEQ ID NO: 1 resulted in higher alpha-santalene levels than in the wildtype, and introducing a Histidine at this position destroyed the activity as a santalene synthase. This demonstrated that the position is important, but it is also important how it is changed.

The I291V, 129S, I291C, I291F and I291T mutants were also tested and showed—as the N267S or N267L—a surplus of beta-santalene, but in comparison to N267S there was more alpha-santalene remaining, albeit less alpha-santalene than the wildtype control (see FIG. 5 and table 1). The largest percentage value for beta-santalene was found when SEQ ID NO: 34 was expressed in the host cells.

Double mutants with N267S or N267T changes at the position corresponding to position 267 of SEQ ID NO: 1 and a Threonine instead of an Isoleucine at the position corresponding to the position 291 of SEQ ID NO: 1 resulted also in improved beta santalene synthases with an excess of beta-santalene, although they showed an intermediate product profile compared to the single mutant improved beta santalene synthase enzymes (see FIG. 5).

Modelling of these mutants showed in the RSMF plot that the N257S single mutants as well as its double mutants with a Serine, Cysteine or Threonine at the position corresponding to 291 of SEQ ID NO:1 show increased flexibility in Helix C and Helix D, which s concurrent with the experimental results for N267S and its double mutant with Threonine at the position corresponding to the position 291 of SEQ ID NO: 1 (see FIG. 5). Interestingly, also the flexibility in some other regions further downstream appears to be improved as well, as the RSMF data showed.

### Software Tools Used

### Homology Models

Homology models were generated using the Schrödinger Prime package (www.schrodinger.com/prime; Schrödinger Release 2020-2: Prime, Schrödinger, LLC, New York, N.Y., 2020; M Jacobson et al., Proteins, 2004, 55, 351-367). Template structures were downloaded from PDB—Protein Data Bank (HM Breman et al., Nucleic Acid Research, 2000, 28, 235-242), the template structure for each homology model generation are indicated in Table 2.

TABLE 2
Template structures used for homology model generation.
For each built homology model the Protein DataBase code
of the template structure used is indicated.

| Homology model | Template structure (PDB code) |
|---|---|
| CiCaSSy | 6A1I |
| CiCaSSy N267L | 6A1I |
| CiCaSSy N267S | 6A1I |
| SaSSy | 5ZZJ |
| SaSSy 14 | 5ZZJ |
| ClaSSy | 6A1I |
| SaSSy 134 | 5ZZJ |

### MD Simulations

MD simulations were performed by using the software GROMACS version 2018 (www.gromacs.org; D van Der Spoel et al., J Comput Chem, 2005, 26, 1701-1718). All the enzymes were defined in OPLS-AA forcefield (WL Jorgensen and J Tirado-Rives, J Am Chem Soc, 1988, 110, 1657-1666), enzyme protonation was defined at pH 8.0 and calculated using the tool pdb2pqr (T J Dolinsky et al., Nucleic Acids Res, 2007 35, W522-W525); the 3 metal ions (Mg2+) were included in the model by fixing their relative position to their coordinating amino acid residues as described in MW van der Kamp et al., Biochemistry, 2013, 52, 8094-8105. Each enzyme was put in the center of a cubic system of 1000 nm3 and explicitly solvated with TIP4P water (WL Jorgensen et al., J Chem Phys, 1983, 79, 926-935), total charge of the system was neutralized by adding the opportune amount of Na+ or Cl− ions. Each system was minimized for 10000 steps, using a steepest descent algorithm and subsequently equilibrated for 10 ns. After equilibration, each system was simulated for 500 ns using. Temperature was kept constant at 300 K using the v-rescale algorithm (G Bussi et al., J Chem Phys, 2007, 126, 014101), pressure was kept constant at 1 atm using the Parrinello-Rahman algorithm (M Parrinello and A Rahman, Phys Rev Lett, 1980, 45, 1196-1198.) and electrostatic interactions were simulated by the extended particle mesh Ewald algorithm (U Essmann et al., J Chem Phys, 1995, 103, 8577-8593). Simulation frames were saved every 5 ps.

### RMSD

Root Mean Square Deviation (RMSD) was evaluated for each enzyme structure on the full simulation length (500 ns). Calculations were performed by the gmx rms tool of the GROMACS package after having performed a structural superimposition of the protein structure for each trajectory frame (gmx trjconv) using the equilibrated system as a reference.

### RMSF

Root Mean Square Fluctuation (RMSF) was evaluated for each enzyme structure on the last 450 ns of simulation. Calculations were performed by the gmx rmsf tool of the GROMACS package after having performed a structural superimposition of the protein structure for each trajectory frame (gmx trjconv) and using the protein Cα of the equilibrated system as a reference.

### Pictures

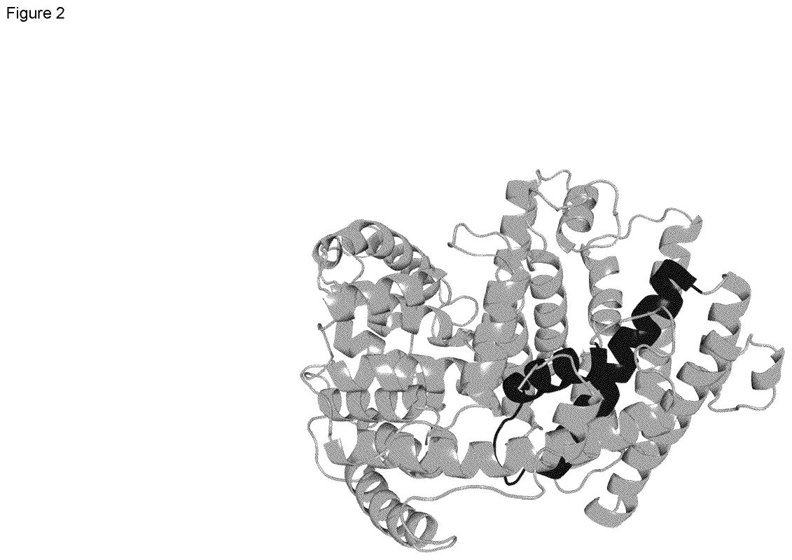

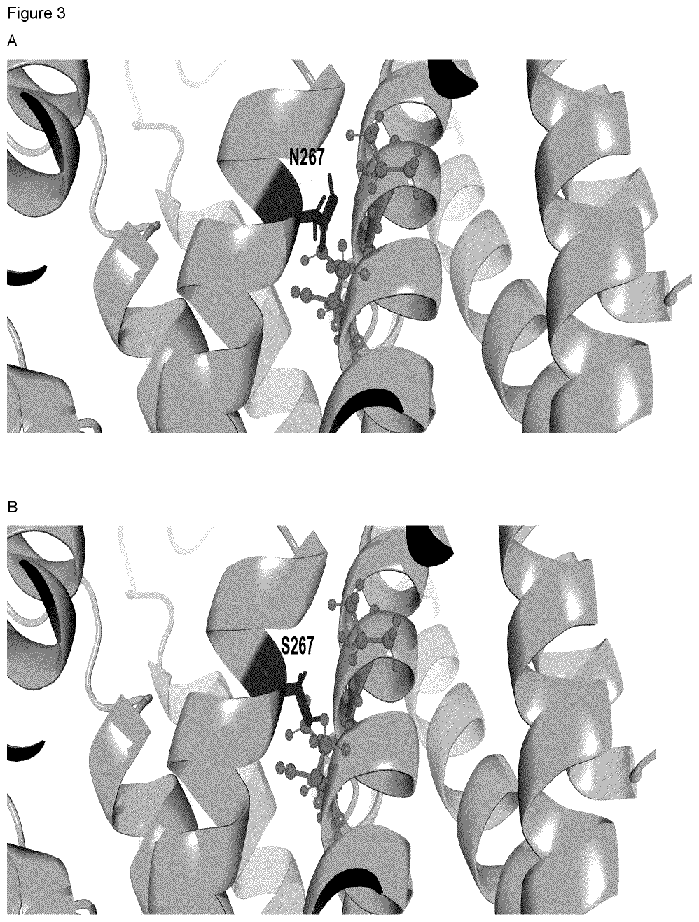

The protein pictures for FIGS. 2 and 3 were generated by using the PyMOL (pymol.org) software. RMSD and RMSF pictures were generated by using the Matplotlib library (matplotlib.org) for Python version 3.6 (python.org).

### PFAM Domain Analysis

PFAM domain PF01397 “Terpene_synth” and a C-terminal PFAM domain PF03936 “Terpene_synth_C” were identified using version 32.0 of the PFAM software on May 29, 2020 and confirmed with version 33.1 of the PFAM software released on Jun. 11, 2020; for details on PFAM see “The Pfam protein families database in 2019: S. EI-Gebali, J. Mistry, A. Bateman, S. R. Eddy, A. Luciani, S. C. Potter, M. Qureshi, L. J. Richardson, G. A. Salazar, A. Smart, E. L. L. Sonnhammer, L. Hirsh, L. Paladin, D. Piovesan, S. C. E. Tosatto, R. D. Finn Nucleic Acids Research (2019) and http://pfam.xfam.org/ and “Pfam: The protein families database in 2021: J. Mistry, S. Chuguransky, L. Williams, M. Qureshi, G. A. Salazar, E. L. L. Sonnhammer, S. C. E. Tosatto, L. Paladin, S. Raj, L. J. Richardson, R. D. Finn, A. Bateman Nucleic Acids Research (2020) doi: 10.1093/nar/gkaa913”

### Interpro Motifs

The following domains

| | |
|---|---|
| “Terpene synthase, metal-binding domain” | IPR005630 |
| “Terpene cyclase-like 1, C-terminal domain” | IPR034741 |
| “Terpene synthase, N-terminal domain” | IPR001906 |
| And these homologous superfamilies | |
| “Isoprenoid synthase domain superfamily” | IPR008949 |
| “Terpenoid cyclases/protein prenyltransferase alpha-alpha toroid” | IPR008930 |
| “Terpene synthase, N-terminal domain superfamily” | IPR036965 |

were identified with the InterPro scan software version 83.0, released December 2020; for further details of InterPro see: Blum M, Chang H, Chuguransky S, Grego T, Kandasaamy S, Mitchell A, Nuka G, Paysan-Lafosse T, Qureshi M, Raj S, Richardson L, Salazar G A, Williams L, Bork P, Bridge A, Gough J, Haft D H, Letunic I, Marchler-Bauer A, Mi H, Natale D A, Necci M, Orengo C A, Pandurangan A P, Rivoire C, Sigrist C J A, Sillitoe I, Thanki N, Thomas P D, Tosatto S C E, Wu C H, Bateman A and Finn R D The InterPro protein families and domains database: 20 years on. Nucleic Acids Research, November 2020, (doi: 10.1093/nar/gkaa977)

1.-15. (canceled)

16. A synthetic beta santalene synthase characterized by the fact that the tertiary structure of the part of said synthetic beta santalene synthase that correspond to the stretch from amino acid positions 272 to position 291 of SEQ ID NO: 1 has an increased flexibility compared to the flexibility of the same tertiary structure in a naturally occurring santalene synthase, wherein the flexibility is determined by root mean square fluctuation analysis using simulations for 500 ns for both the synthetic and the naturally occurring santalene synthase with these settings: pH 8.0, 300 K, 1 atm, water environment, ions present without substrate, and evaluation for each enzyme structure on the last 450 ns of simulation; and wherein said synthetic beta santalene synthase is further characterized by its ability to produce beta-santalene and alpha-santalene in a ratio that is equal to or greater than 1 under typical conditions suitable for the production of both these santalenes, wherein beta-santalene is (−)-β-santalene (CAS number 511-59-1).

17. A synthetic beta santalene synthase producing beta-santalene and alpha-santalene from farnesyl pyrophosphate, wherein the santalene synthase has at least 50% sequence identity to
a. the amino acid positions 261 to 278, preferably position 261 to position 272, of SEQ ID NO: 2, 3, 29, 57 or 58 wherein the position corresponding to position 261 of SEQ ID NO. 2, 3, 29, 57 or 58 is an Arginine residue and the position corresponding to position 278 of SEQ ID NO: 2, 3, 29, 57 or 58 is a Proline residue and said Arginine and said Proline are used to align the two protein sequences for the sequence identity determination; or
b. the amino acid positions 261 to 302 of SEQ ID NO: 2, 3 or 40, wherein the position corresponding to position 261 of SEQ ID No. 2 or 3 is an Arginine residue and three Aspartate residues are found at positions that correspond to the Aspartates at positions 298, 299 and 302 of SEQ ID NO: 2 or 3 or 29 to 40; or
c. a combination of a. and b. above; or
d. the full-length of SEQ ID NO: 1
e. a combination of any of a. to c. above with d.;
and wherein said synthetic beta santalene synthase is producing beta-santalene and alpha-santalene in a ratio of the two that is equal to or greater than 1 under conditions suitable for the production of these santalenes.

18. The synthetic beta santalene synthase of claim 16, wherein it further has the following amino acids at the position corresponding to the positions in SEQ ID NO 1 provided in brackets:
An Arginine (261), Aspartate (262), Arginine (263), Leucine or Isoleucine or Valine or Methionine (264) Leucine or Isoleucine or Valine (265), Glutamic Acid or Glutamine (266) and Histidine or Tyrosine (268).

19. The synthetic beta santalene synthase of claim 16 that has at the amino acid position corresponding to
a. position 267 of SEQ ID NO: 1 any of the following amino acids:
Serine, Leucine, Threonine, Cysteine, Isoleucine, Valine, Tryptophan, Glycine or Alanine; or
b. position 291 of SEQ ID NO: 1 any of the following amino acids:
Threonine, Cysteine, Serine, Phenylalanine or Valine; or
c. a combination of a. and b. above; or
d. position 267 of SEQ ID NO: 1 an Asparagine and the position corresponding to position 291 of SEQ ID NO: 1 is any of the following amino acids:
Threonine, Cysteine, Serine, Phenylalanine or Valine; or
e. position 267 of SEQ ID NO: 1 any of the following amino acids:
Serine, Leucine, Threonine, Cysteine, Isoleucine, Valine, Tryptophan, Glycine or Alanine and the position corresponding to position 291 of SEQ ID NO: 1 is an Isoleucine.

20. A synthetic nucleic acid encoding for the synthetic santalene synthase of claim 16.

21. An expression cassette comprising the synthetic nucleic acid of claim 20.

22. A method for the production of a composition comprising beta-santalene in excess of alpha-santalene comprising the following steps:
I. providing one or more improved beta santalene synthase according to claim 16 in an active form and with all required co-factors,
II. contacting farnesyl pyrophosphate with the one or more improved beta santalene synthases under conditions permitting the production of santalenes,
III. producing beta-santalene and alpha santalene and optionally bergamotene from farnesyl pyrophosphate, wherein the amount of beta-santalene produced is larger than the amount of alpha-santalene produced, wherein beta-santalene is (−)-β-santalene (CAS number 511-59-1),
IV. optionally purifying the products.

23. A non-human host cell suitable to produce the santalene synthase according to claim 16 from a nucleic acid encoding said santalene synthase and suitable to provide the santalene synthase with farnesyl pyrophosphate and all co-factors required for its activity, wherein the host cell comprises a nucleic acid encoding the santalene synthase.

24. The santalene synthase, non-human host cell or the method of claim 16 wherein the santalene synthase produces an excess of trans-α-bergamotene over alpha-santalene in addition to producing more beta-santalene than alpha-santalene.

25. A composition produced by a synthetic santalene synthase, the method or the non-human host cell of claim 16, wherein the composition comprises beta-santalene in excess to alpha-santalene, wherein beta-santalene is (−)-β-santalene (CAS number 511-59-1).

26. The composition of claim 25, wherein the composition comprises more beta-santalene than bergamotene, and more bergamotene than alpha-santalene.

27. The composition of claim 16 wherein the composition comprises at least 12% (w/w) trans-α-bergamotene.

28. A method for the production of a composition comprising beta-santalol in excess to alpha-santalol comprising the steps of:
I. Producing a composition comprising beta-santalene in excess over alpha-santalene by a method of claim 16 or with the host cells or the santalene synthases of claim 16;
II. Oxidising at least some of the beta-santalene and alpha-santalene in the composition produced in a) to their respective alcohols to produce a composition comprising beta-santalol in excess of alpha-santalol; and
III. optionally purifying the products.

29. A composition produced by the method of claim 28 comprising beta-santalol in excess to alpha-santalol, wherein the beta-santalol to alpha santalol ratio is at least 1.3.

30. Use of any of the synthetic santalene synthases of SEQ ID NO: 2, 3, 13 to 53, 56 to 58 for the production of a composition comprising alpha-santalene, beta-santalene and trans-a-bergamotene.


SEQUENCE LISTING (58 sequences)

SEQ ID NO: 1 - protein, 553 aa, Cinnamomum camphora
  wildtype CiCaSSy
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLENHLFSIAVVVEPQYSQCRVAITKAIVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 2 - protein, 553 aa, Artificial Sequence
  >CiCaSSy_N267S
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLESHLFSIAVVVEPQYSQCRVAITKAIVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 3 - protein, 553 aa, Artificial Sequence
  >CiCaSSy_N267L
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLELHLFSIAVVVEPQYSQCRVAITKAIVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 4 - protein, 569 aa, Santalum album
  >SaSSy
MDSSTATAMTAPFIDPTDHVNLKTDTDASENRRMGNYKPSIWNYDFLQSLATHHNIVEERHLKLAEKLKGQVKFMFGAPMEPLAKLELVDVVQRLGLNHLFETEIKEALFSIYKDGSNGWWFGHLHATSLRFRLLRQCGLFIPQDVFKTFQNKTGEFDMKLCDNVKGLLSLYEASYLGWKGENILDEAKAFTTKCLKSAWENISEKWLAKRVKHALALPLHWRVPRIEARWFIEAYEQEANMNPTLLKLAKLDFNMVQSIHQKEIGELARWWVTTGLDKLAFARNNLLQSYMWSCAIASDPKFKLARETIVEIGSVLTVVDDGYDVYGSIDELDLYTSSVERWSCVEIDKLPNTLKLIFMSMFNKTNEVGLRVQHERGYNSIPTFIKAWVEQCKSYQKEARWFHGGHTPPLEEYSLNGLVSIGFPLLLITGYVAIAENEAALDKVHPLPDLLHYSSLLSRLINDIGTSPDEMARGDNLKSIHCYMNETGASEEVAREHIKGVIEENWKILNQCCFDQSQFQEPFITFNLNSVRGSHFFYEFGDGFGVTDSWTKVDMKSVLIDPIPLGEE

SEQ ID NO: 5 - protein, 569 aa, Santalum album
  >SaSSy14
MDSSTATAMRAPFIDHTDHVNLRTDNDSSENRRMGNYKPSIWNYDFLQSLATRHNIMEERHLKLAEKLKGQVKFMFGAPMEPLAKLELVDVVQRLGLNHRFETEIKEALFSIYKDESNGWWFGHLHATSLRFRLLRQCGLFIPQDVFKTFQSKTGEFDMKLCDNVKGLLSLYEASFLGWRDENILDEAKAFATKYLKNAWENISQKWLAKRVKHALALPLHWRVPRIEARWFVEAYGEEENMNPTLLKLAKLDFNMVQSIHQKEIGELARWWVTTGLDKLAFARNNLLQSYMWSCAIASDPKFKLARETIVEIGSVLTVVDDAYDVYGSMDELDLYTNSVERWSCTEIDKLPNTLKLIFMAMFNKTNEVGLRVQHERGYSGITTFIKAWVEQCKSYQKEARWYHGGHTPPLEEYSLNGLVSIGFPLLLITGYVAIAENEAALDKVHPLPDLLHYSSLLSRLINDMGTSSDELERGDNLKSIQCYMNQTGASEKVAREHIKGIIEENWKILNECCFDQSQFQEPFVTFNLNSVRGSHFFYEFGDGFGVTNSWTKVDMKSVLIDPIPLDEE

SEQ ID NO: 6 - protein, 569 aa, Santalum spicatum
  >SpiSSy
MDSSTATATTAPFIDHTDHVNLKIDNDSSESRRMGNYKPSIWNYDFLQSLAIHHNIVEEKHLKLAEKLKGQVMSMFGAPMEPLAKLELVDVVQRLGLNHQFETEIKEALFSVYKDGSNGWWFGHLHATSLRFRLLRQCGLFIPQDVFKTFQSKTDEFDMKLCDNIKGLLSLYEASFLGWKGENILDEAKAFATKYLKNAWENISQKWLAKRVKHALALPLHWRVPRIEARWFIEAYEQEENMNPTLLKLAKLDFNMVQSIHQKEIGELARWWVTTGLDKLAFARNNLLQSYMWSCAIASDPKFKLARETIVEIGSVLTVVDDAYDVYGSMDELDHYTYSVERWSCVEIDKLPNTLKLIFMSMFNKTNEVGLRVQHERGYNGIPTFIKAWVEQCKAYQKEARWYHGGHTPPLEEYSLNGLVSIGFPLLLITGYIAIAENEAALDKVHPLPDLLHYSSLLSRLINDMGTSPDEMARGDNLKSIHCYMNETGASEEVAREHIKGIIEENWKILNQCCFDQSQFQEPFITFNLNSVRGSHFFYEFGDGFGVTDSWTKVDMKSVLIDPIPLGEE

SEQ ID NO: 7 - protein, 569 aa, Santalum austrocaledonicum
  >SauSSy
MDSSTATAMTAPFIDPTDHVNLKTDTDASENRRMGNYKPSIWNYDFLQSLATHHNIVEERHLKLAEKLKGQVKFMFGAPMEPLAKLELVDVVQRLGLNHRFETEIKEALFSIYKDESNGWWFGHLHATSLRFRLLRQCGLFIPQDVFKTFQNKTGEFDMKLCDNVKGLLSLYEASYLGWKGENILDEAKAFATKYLKSAWENISEKWLAKRVKHALALPLHWRVPRIEARWFIEAYEQEANMNPTLLKLAKLDFNMVQSIHQKEIGELARWWVTTGLDKLAFARNNLLQSYMWSCAIASDPKFKLARETIVEIGSVLTVVDDAYDVYGSMDELDLYTSSVERWSCVEIDKLPNTLKLIFMSMFNKTNEVGLRVQHERGYNSIPTFIKAWVQQCKSYQKEARWFHGGHTPPLEEYSLNGLVSIGFPLLLITGYVAIAENEAALDKVHPLPDLLHYSSLLSRLINDIGTSPDEMARGDNLKSIHCYMNGTGASEEVAREHIKGVIEENWKILNQCCFDQSQFQEPFITFNLNSVRGSHFFYEFGDGFGVTDSWTKVDMKSVLIDPIPLGEE

SEQ ID NO: 8 - protein, 551 aa, Clausena lansium
  >ClaSSy
MSTQQVSSENIVRNAANFHPNIWGNHFLTCPSQTIDSWTQQHHKELKEEVRKMMVSDANKPAQRLRLIDTVQRLGVAYHFEKEIDDALEKIGHDPFDDKDDLYIVSLCFRLLRQHGIKISCDVFEKFKDDDGKFKASLMNDVQGMLSLYEAAHLAIHGEDILDEAIVFTTTHLKSTVSNSPVNSTFAEQIRHSLRVPLRKAVPRLESRYFLDIYSRDDLHDKTLLNFAKLDFNILQAMHQKEASEMTRWWRDFDFLKKLPYIRDRVVELYFWILVGVSYQPKFSTGRIFLSKIICLETLVDDTFDAYGTFDELAIFTEAVTRWDLGHRDALPEYMKFIFKTLIDVYSEAEQELAKEGRSYSIHYAIRSFQELVMKYFCEAKWLNKGYVPSLDDYKSVSLRSIGFLPIAVASFVFMGDIATKEVFEWEMNNPKIIIAAETIFRFLDDIAGHRFEQKREHSPSAIECYKNQHGVSEEEAVKALSLEVANSWKDINEELLLNPMAIPLPLLQVILDLSRSADFMYGNAQDRFTHSTMMKDQVDLVLKDPVKLDD

SEQ ID NO: 9 - protein, 567 aa, Artificial Sequence
  >SaSSY134
MDSSTATATTAPFIDPTNHVNLKIDNDSSENRRMGNYKPSIWNYDFLQSLATHHNIVEERHLKLAEKLKGQVRILLKEKMEPLAQLELVDVVQRLGLNHLLETEIKEALFSIYKDHIDSDKADLHATSLRFRLLRQQGIKISCDVFEQFKDDEDRFKSSLINDIQGMLSLYEASFLGWKGENILDEAKAFATKYLKAMVESLGGHLAKRVKHALALPLHWRVPRIEARWFIEAYEQEENMNPTLLKLAKLDFNMVQSIHQKEIGELARWWVTTGLDKLAFARNNLLQSYMWSCAIASDPKFKLARETIVEIGSVLTVVDDAYDVYGHMDELDLYTSSVEGWSCAEIDRLPDTLKLIFMSMFNKTNEVGLRVQHERGYNSIPTFIKAWVEQCKSYQKEARWFHGGHTPPLEEYSLNGLVSIGFPLLLITGYIAIAENEAALDKVRPLPDLLHYSSLLSRLINDMGTSPDEMARGDNLKSIHCYMNETGASEEVAREHIKGIIEENWKILNQCCFDQSQFQEPFITFNLNSVRGSHFFYEFGDGFGVTDSWTKVDMKSVLIDPIPLGEE

SEQ ID NO: 10 - DNA, 1662 bp, Cinnamomum camphora
  >CiCaSSy_wt
atggacagcatggaagtccggcggtcggcgatctaccacagcacgttctgggacatcgacagcatccgggcgctcctggcgcggcgggactgcacggcggccgcggccctctcgcccgaccaccataagcgcctgaaggagcgcatccagcgccgcctccaggacatcacccagccccaccatctgctcggcctcatcgacgccgtgcagcgcctgggcgtggcctaccagttcgaggaagagatctcggacgcgctgcacggcctccattcggagaacaccgagcacgccatcaaggactcgctgcaccatacgtcgctctatttccgcctgctccgccagcatggctgcaacctgtcgtcggacatcttcaacaagttcaagaaggaaggcggcggcttcaaggcctcgctctgcgaggacgccatgggcctgctctcgctgtatgaggccgtgcgcctctcggtgaagggcgaggccatcctggaggaagcccaggtgttctcgatcgccaacctgaagatcctcatggagcgcgtggagcgcaagctcgccgaccgcatcgagcatgccctggagatcccgctctattggcgcgccccgcgtctggaggcccgctggtacatcgacgtgtatgagaaggaagacggccgcatcgacgacctgctcgacttcgcgaagctggacttcaaccgcgtgcagatgctctatcagaccgagctgaaggagctctcgatgtggtgggagctgctgggcctgcccgccaagatgggcttcttccgcgaccgcctgctcgagaaccacctcttctcgatcgccgtggtggtggagccccagtactcgcagtgccgcgtggccatcaccaaggcgatcgtgctgatgacggcgatggacgacttctatgacgtgcatggcctgccggacgagctcaaggtgttcaccgacacggtgaaccgctgggacctggagggcatcgaccagctccccgagtacatgaagctgtactatctggcgctctacaacaccacgaacgagacggcctatatcatcctgaaggagaagggcttcaacgccacgcattacctgaagaagctctgggccatgcagtcgaacgcgtatttccgcgaggcccagtggttcaactcgggctacatcccgaagttcgacgagtatctggacaacgccctcgtgtcggtgggcgccccgttcgtgctgggcctctcgtatcccatgatccagcagcagatctcgaaggaagagatcgacctgatccccgaggacctcaacctgctccgctgggcctcgatcatcttccgcctgtacgacgacctggccacctcgaaggccgagcagcagcgcggcgacgtgcccaagtcgatccagtgctatatgcatgagacgggctcgtcggaggaagtggcggccaaccatatccgcgacctgatctcggacgcgtggaaggaagtgaacgccgagtgcctgaagccgacctcgctctcgaagcactacgtgggcgtggcccccaactcggcccgctcgggcgtgctcatgtatcaccatgacttcgacggcttcgcgtcgccccatggccgcacgaacgcccacatcacgagcatcttcttcgagccggtccccctcaaggagagcatcaacctgggctga

SEQ ID NO: 11 - DNA, 1662 bp, Artificial Sequence
  >CiCaSSy_N267S
atggacagcatggaagtccggcggtcggcgatctaccacagcacgttctgggacatcgacagcatccgggcgctcctggcgcggcgggactgcacggcggccgcggccctctcgcccgaccaccataagcgcctgaaggagcgcatccagcgccgcctccaggacatcacccagccccaccatctgctcggcctcatcgacgccgtgcagcgcctgggcgtggcctaccagttcgaggaagagatctcggacgcgctgcacggcctccattcggagaacaccgagcacgccatcaaggactcgctgcaccatacgtcgctctatttccgcctgctccgccagcatggctgcaacctgtcgtcggacatcttcaacaagttcaagaaggaaggcggcggcttcaaggcctcgctctgcgaggacgccatgggcctgctctcgctgtatgaggccgtgcgcctctcggtgaagggcgaggccatcctggaggaagcccaggtgttctcgatcgccaacctgaagatcctcatggagcgcgtggagcgcaagctcgccgaccgcatcgagcatgccctggagatcccgctctattggcgcgccccgcgtctggaggcccgctggtacatcgacgtgtatgagaaggaagacggccgcatcgacgacctgctcgacttcgcgaagctggacttcaaccgcgtgcagatgctctatcagaccgagctgaaggagctctcgatgtggtgggagctgctgggcctgcccgccaagatgggcttcttccgcgaccgcctgctcgagtcgcacctcttctcgatcgccgtggtggtggagccccagtactcgcagtgccgcgtggccatcaccaaggcgatcgtgctgatgacggcgatggacgacttctatgacgtgcatggcctgccggacgagctcaaggtgttcaccgacacggtgaaccgctgggacctggagggcatcgaccagctccccgagtacatgaagctgtactatctggcgctctacaacaccacgaacgagacggcctatatcatcctgaaggagaagggcttcaacgccacgcattacctgaagaagctctgggccatgcagtcgaacgcgtatttccgcgaggcccagtggttcaactcgggctacatcccgaagttcgacgagtatctggacaacgccctcgtgtcggtgggcgccccgttcgtgctgggcctctcgtatcccatgatccagcagcagatctcgaaggaagagatcgacctgatccccgaggacctcaacctgctccgctgggcctcgatcatcttccgcctgtacgacgacctggccacctcgaaggccgagcagcagcgcggcgacgtgcccaagtcgatccagtgctatatgcatgagacgggctcgtcggaggaagtggcggccaaccatatccgcgacctgatctcggacgcgtggaaggaagtgaacgccgagtgcctgaagccgacctcgctctcgaagcactacgtgggcgtggcccccaactcggcccgctcgggcgtgctcatgtatcaccatgacttcgacggcttcgcgtcgccccatggccgcacgaacgcccacatcacgagcatcttcttcgagccggtccccctcaaggagagcatcaacctgggctga

SEQ ID NO: 12 - DNA, 1662 bp, Artificial Sequence
  >CiCaSSy_N267L
atggacagcatggaagtccggcggtcggcgatctaccacagcacgttctgggacatcgacagcatccgggcgctcctggcgcggcgggactgcacggcggccgcggccctctcgcccgaccaccataagcgcctgaaggagcgcatccagcgccgcctccaggacatcacccagccccaccatctgctcggcctcatcgacgccgtgcagcgcctgggcgtggcctaccagttcgaggaagagatctcggacgcgctgcacggcctccattcggagaacaccgagcacgccatcaaggactcgctgcaccatacgtcgctctatttccgcctgctccgccagcatggctgcaacctgtcgtcggacatcttcaacaagttcaagaaggaaggcggcggcttcaaggcctcgctctgcgaggacgccatgggcctgctctcgctgtatgaggccgtgcgcctctcggtgaagggcgaggccatcctggaggaagcccaggtgttctcgatcgccaacctgaagatcctcatggagcgcgtggagcgcaagctcgccgaccgcatcgagcatgccctggagatcccgctctattggcgcgccccgcgtctggaggcccgctggtacatcgacgtgtatgagaaggaagacggccgcatcgacgacctgctcgacttcgcgaagctggacttcaaccgcgtgcagatgctctatcagaccgagctgaaggagctctcgatgtggtgggagctgctgggcctgcccgccaagatgggcttcttccgcgaccgcctgctcgagctccacctcttctcgatcgccgtggtggtggagccccagtactcgcagtgccgcgtggccatcaccaaggcgatcgtgctgatgacggcgatggacgacttctatgacgtgcatggcctgccggacgagctcaaggtgttcaccgacacggtgaaccgctgggacctggagggcatcgaccagctccccgagtacatgaagctgtactatctggcgctctacaacaccacgaacgagacggcctatatcatcctgaaggagaagggcttcaacgccacgcattacctgaagaagctctgggccatgcagtcgaacgcgtatttccgcgaggcccagtggttcaactcgggctacatcccgaagttcgacgagtatctggacaacgccctcgtgtcggtgggcgccccgttcgtgctgggcctctcgtatcccatgatccagcagcagatctcgaaggaagagatcgacctgatccccgaggacctcaacctgctccgctgggcctcgatcatcttccgcctgtacgacgacctggccacctcgaaggccgagcagcagcgcggcgacgtgcccaagtcgatccagtgctatatgcatgagacgggctcgtcggaggaagtggcggccaaccatatccgcgacctgatctcggacgcgtggaaggaagtgaacgccgagtgcctgaagccgacctcgctctcgaagcactacgtgggcgtggcccccaactcggcccgctcgggcgtgctcatgtatcaccatgacttcgacggcttcgcgtcgccccatggccgcacgaacgcccacatcacgagcatcttcttcgagccggtccccctcaaggagagcatcaacctgggctga

SEQ ID NO: 13 - protein, 553 aa, Artificial Sequence
  >VarS1
MDSMEVRRTAIYHSTFWDIDSIRALIARRDCTAAAAISPDHHKRLKERIQKRLQDITQPHHLLGLIDAVQRLGVAYQFEEEISDALHGLHSENTEHGIRDSLHHTSLYFRLLRQHGCNLSSDIFNKFKKDGGGFKATLCDDAMGLLSLYEAVRLSVKGEAILEEAQVFSIANLKILLDRLDRKLAERIEHALDLPLFWRAPRLEARWYIDLYEKDDGRIDDLLDFAKLDFNRVQLIYQTELKELSMWWELVGLPAKMGFFRDRILESHLFTVAIVVEPQYSQCRLAITKALILMTAMDDFYDVHGLPDELKLFTDTVQRWDIEAMEQLPEYMKIYYLALYNTTNETAYVILKEKGFNATHYLKKVWAMQSNAYFREAQWFNSGYVPKFDEYLDNALVSVGAPFVLGVTYPVIQQQLSKEELDLVPEDLNLVKWASIIFRLYDDLATSKAEQQRGDVPKSIQCYMHETGSSEEVAANHIRDMISDAWKDVNAECLKPTSLSKHYLGLAPNSARSGVLLYHHDFDGFATPHARTNGHITSIFFDPVPLKESINLG

SEQ ID NO: 14 - protein, 553 aa, Artificial Sequence
  >VarS2
MDSVDMRKSAIYHSTFWDIDSIRALLARRDCTAAAALSPEHHKRLKERIQKRLNEVTQPHHLLGLIDAVQRLGVAYQFEEEISEALHGIHSENTEHGVRESLHHTSLYFRLLRQHGCNLSSDIFNRFKKDGGGFKGSLCEDAMGLLSLYEAVRLSLKGEAILEEAQVFSIAQLRILMERVEKKLAERIEHALDIPLYWRAPRLEARWYIEVYEKEEGKIEDLLDFAKLDFNRVQMLYQSELKELSMWWELMGLPAKMGFFRDRLLESHLFSMAVVVEPQYSQCRVAITKAIVLMSAMDDFYDVHGLPDELKVFTDTVQRWDVEGMDQLPDYMKLYYLALYNTTNETAYVILREKGFNATHYLKKLWAMQSNAYFKEAQWFNSGYVPKFDEYLDNALVSIGGPFVLALSYPLLQQQISKEEIELIPEDLNLIRWASIIFRLYDDLATTKAEQNRGDVPKSIQCYMHETGSSEEVAANHIRDLISDAWKDVNAECLKPSSLSKHYLGVAPNTARTGVLMYHHDFDGFASPHGKTNAHITSIFFEPVPMKESINLG

SEQ ID NO: 15 - protein, 553 aa, Artificial Sequence
  >VarS3
MDSMEVRKSAMYHSTFWDIDSIRAILAKRDCTAAAALSPEHHKRLKERIQKRLNDVTNPHHLLALIDAVQRLGVAYQFEEEISDALHGLHSEQSDHGLKDSLHHTSLYFRLLRQHGCNLSSDIFNKFKKEGGGFKATLCEDAMGLLSLYEAVRLSIRGEAILEEAQIFSIANLKIIIERLERKLAEKIEHALEIPLYWRAPRLEARWYIEIYEKDDGKIEDLLDFAKLDFNRMQMLYQTELKDLTMWWELLGLPAKMGFFRDRLLETHLFSIAMIVEPQYSNCRIGITKAIVLVSAMDDFYDVHGLPDELRVFTETVNRWDLEGIDQLPDYMKVYYLGLYNTTNETAYLVVKDKGYNATHYLRKVWAMQSNAYFREAQWFNTGYIPKFDEYLDNAVVSVGAPFVVGLTYPLLQQQLTKEEIELIPEDLNLLRWASVIFRLYDDLATTKAEQQRGDMPKSIQCYMHETGTSEEVAANHIKEIISEAWKDVNAECLKPSSVSKHYLGLAPNSARSAMVIYHHDFDGFATPHGKTNAHITSIFFDPVPIKDSINLG

SEQ ID NO: 16 - protein, 553 aa, Artificial Sequence
  >VarS4
MDSVEIRRSAIYHSTFWDIDSIRALIARRDCSGGGALSPDHHKRLKEKIQRRLNDVTNPHHLLGLIDAVQRLGVAYQFEEEISEALHGMHSENSDHGVRESLHHTSLYFRLLRQHGCNLSSDIFQRFRKDGGGFRASICDDAMGLLSLYEAVRLSIRGEAILEEAQLFSIANLKLVVDKVEKKLADRIEHALDLPIYWRAPRLEARWYIDIYERDEARIEDLLDFAKLDFNRVQMLYQTELKDLTMWWDLLGLPAKMGFFRDRLLETHLFSLALIVEPQFSNCRMAMTKAVILISAMDDFYDIHGLPDELKIFTETVNRWDVEGVDQLPEYMKIYYVALFNTTNETAYLMLKDKGYNATHFLKKIWAIQSNAYFREAQWYNSGYIPKFDEYLENAVISVGGPFLVGLSYPVMQNNITKEDIDLVPEDLNLLKWASMIFRLWDDLGTSKAEQQRGEVPKSIQCYMHETGSSEEVAANHIKEMITDAWKDVNGECLKPTTLSRHYMGIAPNSARTAVVVYHHDFDGFATPHGRSNAHITSIFFDPVPIKESINLG

SEQ ID NO: 17 - protein, 553 aa, Artificial Sequence
  >VarS5
MDSIDIRRTAIYHSTFWDIDSIRALLAKRECSAAGAMSPDHHKKLKEKIQKKLQELTQPHHLLALIDAVQRLGVAYQFEDEISEALHGIHSDNSEHAIRESLHHTSLYFRLLRQHGCNLSTDIFNKFRKDGGGFKATLCEDAMGLLSLYEAVRLSVRGEAILEEAQIFSIANLRIVIEKIERKLADKIEHALELPLYWRAPRLEARWYIDIYEKDEGRIEDLLDFAKLDFNRVQLIYQTELRELTMWWELLGLPAKMGFFRDRLLESHLFSVAMLVEPNYSQCRVALTKAVVLVSAMDDFYDIHGLPDELRLFTETVQRWDLDALDQLPEYMKIFYMALYNTTNETAYLMLKDKGFNASHYLKKVWALQSNGYFREAQWYNSGYVPKFDEYLDNAVISVGGPFLLGVSYPLLQNQLTKEEMELLPEDLNLVKFASVIFRLYDDLATSKAEQNRGDLPKSIQCYMHETGSSEEMAAQHIKDIISEGWKDVNAECLRPSSVSRHFLGLAPNSARSGVIVYHHDFDGFASPHARSNAHITSIFFEPVPIRESINLG

SEQ ID NO: 18 - protein, 553 aa, Artificial Sequence
  >VarS6
MDSMEIRRTGIYHSTFWDIESIKALLAKRDCSAAGAVSPEHHKKLKERIQKKLQDVTQPHHVLGLIDAVQRLGVAYQFDEEITEALHGIHSENTDHGVRDSLHHTSLYFRLLRQHGCNLSSDIFNKFKKDGGGFRATLCDDAMGLLSLYEAVRLSVRGEAILEEAQIFSIANLRIMLERMDKKLAERIEHALEVPIFFRAPRLEARFYIEIYERDEGRMEDLLDFAKLDFNRLQVIYQEELKDLTMWWELLGLPAKMGFFRDRVLETHLFTVALMVEPNFSNCRIGLTKALILITAMDDFYDIHGLPDELKLFTETVQRWDIEAVDNLPDYMKIYYLALYNTTQETAYILLKDKGWNATHFLKKIWAIQSQGYYKEAQWYQTGYLPKFDEYLENAVISIGGPFMLALSYPLLQQQLTREEVDLVPEDLNLIKWGSLIFRLYDDLGTSKAENNRGEMPKSIQCYMHETGSSEEVAANHIKDLISEGWKDVNAECLRPTTVSKHFLALAPNTARSAVLLYHHDFDGFASPHAKTNEHISSIFYDPVPIKDSINLG

SEQ ID NO: 19 - protein, 553 aa, Artificial Sequence
  >VarS7
MDSVELRRTAIYHSTFWDVDSIRALLAKKDCSAAGAISPEHHKKLKERIQKRLNELTNPHHLLALIDAVQRLGVAYQFEDEISEALHGIHSEQTDHGVRESLHHTSLWFRLLRQHGCNLSSDIFNKFRKDGGGFRASVCEDAVGLLSLYEAVRLSMKGEAILEEANLFSVANLRLMLDKIDKKLAEKIEHALDLPLFWRAPRLEARYYIEIYERDEARIEELLDFAKLDFNKLQIIYQTELRDLTMWWELLGLPARMGFFRDRVLESHLFTLALIVEPQFSQCRLGLTKALILVSAMDDFYDVHGLPDELRLFTETVQRWDIEALEQLPEYMKIFYIGLYNTTNETAFMVMREKGYNATHYLRKIWGMQSNGYFKEAQWYQTGYVPKFDEYLENAVISIGGPWLLGLSYPLMQNQLTKEDLEIVPDHLQLVKWGSVIFRLWDDLGTSKAENNRGEMPKTIQCYMHETGTSEEVAANHIKEMISEGWKDVNGECLRPSTVSRHYLAMAPNSARSAVVIYHHEFDGFASPHGKSNGHISSIFFDPVPVRDSINLG

SEQ ID NO: 20 - protein, 553 aa, Artificial Sequence
  >VarS8
MDSVEVRKTGMYHSTFWDVDSIKAILAKKECSAGGAMTPEHHKKLKDRIQKRLNEVSQPHHLLALIDAVQRLGVAYQFEDEISEALHGVHSDNSDHGVKESLHHTSLWFRLLRQHGCQLSSDIFQRFKKDGGGFKASMCEDALGLLSLYEAVRLSIKGEGILEEAQVFSIGNLRIMLDKIDKKLAEKIEHALDVPVYWKAPRLEARFYIDIYEKDEARIEDLLDFAKLDFNRVQMLYQTELKDLTMWWDLIGLPAKMGFFRDRMLESHLFTMAIIMEPNYSNCRIALTKALVLVSAMDDFYDVHGLPDELRLFTETVQRWDIDAMENLPDYMKIFYMALFNTSNETAYILVKDKGWNATHYLRKVWAIQSNAYYKEAQWYNSGYVPKFDEYLENAIISIGGPYLMAVSYPVMQNQVSKEDVELLPEELNLVKYATMIFRLWDDLGTTKAENNRGDVPKTIQCYMHESGTSEEIAANHIKEMISEAWKDVNADCLRPSTLSRHFLGLAPNSARTAVVVYHHDFDGFASPHARSNAHISTIFYDPVPLRDSINLG

SEQ ID NO: 21 - protein, 553 aa, Artificial Sequence
  >VarL1
MDSMEIRRTAIYHSTFWDIDSIRAMLARRDCSAGAAVSPDHHKKLKEKIQRRLQDITQPHHLLGLIDAVQRLGVAYQFEDEISEALHGVHSENTEHAIKDSLHHTSLYFRLLRQHGCNLSSDIFNKFKKEGGGFKATLCEDAMGLLSLYEAVRLSIKGEAILEEAQVFSIANLRIMVEKLERKLADRIEHALEIPLYWRAPRLEARFYIEIYEKEDGKIDELLDFAKLDFNRVQMIYQTELKELTMWWELLGLPAKMGFFRDRLLELHLFSMAVIVEPQFSQCRLALTKAIVLMTAMDDFYDVHGLPDELKVFTDTVNRWDLEGMDQLPEYMKLYYLALYNTTNETAYILVKEKGFNATHYLKRVWAMQSNGYYREAQWFNSGYLPKFDEYLDNALVSVGAPFLLGLSYPMMQQQITKEELDLIPEELNLVRWASIIFRLYDDLATSKAEQQRGDVPKSIQCYMHETGSSEEIAANHIKEMISEAWKDVNAECLKPTSVSKHFVGVAPNSARSGVVMYHHDFDGFASPHGRTNAHITSIFFEPVPIRDSINLG

SEQ ID NO: 22 - protein, 553 aa, Artificial Sequence
  >VarL2
MDSVELRRTGIYHSTFWDIDSIRALLAKRDCTAAAAVSPEHHKKLKERIQKRLNDITQPHHLLGLIDAVQRLGVAYQFEEEISEALHGVHSENTDHAIKDSLHHTSLYFRLLRQHGCNLSSDIFNKFKKDGGGFRATLCEDAMGLLSLYEAVRLSVKGEAILEEAQVFSIANLKIVMERIERKLADRIEHALEIPLYWRAPRLEARWYIDVYEKDDGRIDDLLDFAKLDFNRVQMLYQTELKELSMWWELLGLPAKMGFFRDRLLELHLFSIAMLVEPQYSQCRVALTKAVILISAMDDFYDIHGLPDELRVFTDTVNRWDLEGIEQLPDYMKVYYLALYNTTNETAWIILKEKGFNATHYLKKVWAMQSNAYFREAQWFNTGYIPKFDEYLENALVSVGAPFLLGVSYPLLQNQISKEELELLPEDLNLIRWASMIFRLWDDLATSKAEQQRGEVPKTIQCYMHETGSSEEVAANHIRDMISEAWKEVNAECLKPTSVSRHYVGVAPNTARSGLLMYHHDFDGFASPHGKTNAHITSIFFDPVPLKESINLG

SEQ ID NO: 23 - protein, 553 aa, Artificial Sequence
  >VarL3
MDSVEVRRTGIYHSTFWDIDSIRALVARRDCSAGAALSPDHHKRLKERIQRRLQEISQPHHLLGLIDAVQRLGVAYQFEEEISDALHAIHTDQSDHAIRDSLHHTSLYFRLLRQHGCNLSSDIFQKFKKEGGGFKASLCEDAMGLLSLYEAVRLSIKGEAILEEAQVFSIANLRILLERIERKLAEKIEHALELPLYWRAPRLEARWYIEVYEKDDAKIEDLLDFAKLDFNRLQMMYQTELKELSMWWELLGLPARMGFFRDRILELHLFSIAMVVEPNYSQCRIALTKAIILVSAMDDYYDVHGLPDELKIFTDTVNRWDLEGIEQLPDYMKIYYLALYNTTNETAYILLKEKGFNASHYLRKLWAVQSNAYFKEAQWFNSGYVPKFDEYLENALVSIGAPFIVAVTYPMIQNNVTKEEIELLPEDLQLLKWASVIFRLYDDLGTSKAEQQRGEVPKSIQCYMHETGSSEEVAANHIKEIISDAWKEVNAECLRPTTLSRHYVGVAPNSARSAVVIYHHDFDGFATPHAKTQAHITSIFFEPVPIKESINLG

SEQ ID NO: 24 - protein, 553 aa, Artificial Sequence
  >VarL4
MDSMEVRRSGIYHSTFWDIDSIRALLAKRDCSAAAGLSPEHHKKLKEKIQKKLNEISNPHHLLGLIDAVQRLGVAYQFEDEISEALHGIHSDNTDHAVKESLHHTSLYFRLLRQHGCQLSSDIFNKFRKDGGGFRASLCDDAMGLLSLYEAVRLSVKGEAILEEAQLFSIANLKIMLERLEKKLAERIEHALDLPVYWRAPRLEARFYIDIYERDDARLEDLLDFAKLDFNRLQMMYQTELKELSMWWELLGLPAKMGFFRDRLLEIHLFTIAVLMEPQFSQCRIALTKGIVLVSAMDDFYDVHGLPDELKLFTETVNRWDIEAMEQLPEYMKVFYMALYNTSQETAYIVLKDKGYNASHYLKKIWGMQSNAYFKEAQWYNSGYIPKFDEYLENAVVSLGGPFVLALSYPLMQQQITKEDLELVPEDLNLMKWASVIFRLWDDLATSKAEQQRGELPKSINCYMHETGTSEELAANHIKDLISEAWKDVNAECLRPSTVSKHFMGVAPNSARTAVLIYHHEFDGFASPHGKTNAHISTIFFDPVPIKESINLG

SEQ ID NO: 25 - protein, 553 aa, Artificial Sequence
  >VarL5
MDSLDLRRSAIYHSTFWDIDSIKALIAKRDCSAAAAVSPEHHRKLKEKIQKKLQEISQPHHLLGLIDAVQRLGVAYQFEDEISDALHGMHTDQTDHAVKESLHHTSLWFRLLRQHGCNLSSDIFNRFRKEGGGFKASICEDAMGLLSLYEAVRLSIKGEAILEEAQVFSIANLRIMLEKVEKKLADRIDHALELPIFWRAPRLEARWYIEVYERDEGRIEELLDFAKLDFNRVQMVYQTELKELTMWWELVGLPARMGFFRDRILEIHLFSLAILVEPQYSNCRVALTKAIILISAMDDFYDVHGLPDELKLFTETVQRWDIDAIDNLPEYMKLFYVALYNTTNETAWLIVKERGFNATHYLRKVWAMQSNAYFREAQWFQSGYVPKFDEYLENAVISIGGPFILGLSYPLIQNQVTKEEIDLLPEDLNLVKWASVIFRLYDDLGTSKAENQRGDLPKSIQCYMHETGSSEEVAANHIKEMISEAWKDVNAECLRPSTISRHFLGVAPNSARSAVIMYHHDFDGFASPHAKTNAHITSIFYDPVPLRESINLG

SEQ ID NO: 26 - protein, 553 aa, Artificial Sequence
  >VarL6
MDSVEVRRTGMYHSTFWDIDSIRALLAKRECSAGGAMSPEHHKRLKERIQRKLNDVSQPHHILALIDAVQRLGVAYQFDEEISEALHGIHSEQTDHGMRESLHHTSLYFRLLRQHGCNLSSDIFQKFKKEGGGFKASVCDDAMGLLSLYEAVRLSLKGEAILEEAQIFSIAQLRLMLDRIDKKLAERIEHALELPIYYRAPRLEARFYIEIYDKDEGRIEDLLDFAKLDFNRIQMIYQTELRELTMWWELLGLPAKMGFFRDRILELHLFSLALLVEPNFSNCRMGLTKGIMLVTAMDDFYDVHGLPDELKLFTETVNRWDIDALEQLPDYMKMFYVALFNTTNETAYMLLREKGFNATHFLKKLWALQSNGYFKEAQWYNSGYIPKFDEYLENAIMSIGGPFLLALSYPMVQNQVTREDLEMVPEELNLIKWASLIFRLWDDLGTSKAEQQRGDLPKSIQCYMHESGTSDEVAANHIKEIISEAWKEVNAECLRPSTISRHFMALAPNSARSALVVYHHDFDGFATPHAKTNGHISSIFFDPVPVKESINLG

SEQ ID NO: 27 - protein, 553 aa, Artificial Sequence
  >VarL7
MDSLDIRRTGIYHSTFWDVESIKAIIAKKECTAGGAISPEHHKKLKEKIQKRLNEVSQPHHLLGLIDAVQRLGVAYQFDDEISEALHGVHSDNSDHGLRESLHHTSLWFRLLRQHGCNLSSDIFNRFRKDGGGFKASICEDAVGLLSLYEAVRLSIKGEGILEEAQVFSIANLRIIIDKMDKKLAERIEHALELPLYWRAPRLEARYYIEIYERDEGKLEELLDFAKLDFNRLQMVYQTELRDLTMWWDLLGLPAKMAFFRDRILEVHLFTLAILVEPQFSQCRIGVTKAVVLVSAMDDFYDMHGLPDELRMFTETVQRWDVDAVENLPEYMKVYYMALYNSTNETAFIVMKDKGLNATHYLRKIWAMQSNGYYKEAQWYQSGYVPKFDEYLENALISIGGPFVVGMTYPLMQNNVTKEELELVPDELNLVKWATVIFRLYDDLGTTKAENQRGEVPKSIQCYMHESGTSEEIAANHIKEMISEGWKDVNAECLRPSTISRHFLALAPNTARTAVLLYHHDFDGFASPHAKTNGHISSIFFDPVPMRDSINLG

SEQ ID NO: 28 - protein, 553 aa, Artificial Sequence
  >VarL8
MDSVDIRKTGMYHSTFWDIESIKALMARRECSAGAALSPEHHKKLKDKIQKRLNELSNPHHLLGLIDAVQRLGVAYQFEDEISEALHGVHSENTDHALRESLHHTSLWFRLLRQHGCNLSSDIFNKFRKDGGGFKASLCDDAMGLLSLYEAVRLSLRGEAILEEANLFSIAQLRIILEKIDKKLAEKIDHALDLPIYWRAPRLEARFYIEIYERDEGKLEDLLDFAKLDFNRLQVIYQTELKDLTMWWELLGLPAKMGFFRDRVLEVHLFSMALILEPQYSNCRLGVTKAVMLISAMDDYYDMHGLPDELRLFTETVNRWDVEAMEQLPDYMKIFYMALYNSSNETAFIMLREKGFNATHYLRKIWAVQSQGYFREAQWFQTGYVPKFDEYLENAVISIGGPFLIAVSYPLMQNNLTREDVEILPEELQLIKYATVIFRLWDDLGTSKAEQNRGELPKSINCYMHETGSSEELAANHIREIISEAWKDVNADCLRPSTISRHFLGIAPNTARTGVVVYHHDFDGFATPHARTQAHISTIFFDPVPMRESINLG

SEQ ID NO: 29 - protein, 553 aa, Artificial Sequence
  Cicassy N267T
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLETHLFSIAVVVEPQYSQCRVAITKAIVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 30 - protein, 553 aa, Artificial Sequence
  I291V
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLENHLFSIAVVVEPQYSQCRVAITKAVVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 31 - protein, 553 aa, Artificial Sequence
  I291T
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLENHLFSIAVVVEPQYSQCRVAITKATVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 32 - protein, 553 aa, Artificial Sequence
  I291S
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLENHLFSIAVVVEPQYSQCRVAITKASVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 33 - protein, 553 aa, Artificial Sequence
  I291C
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLENHLFSIAVVVEPQYSQCRVAITKACVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 34 - protein, 553 aa, Artificial Sequence
  I291F
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLENHLFSIAVVVEPQYSQCRVAITKAFVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 35 - protein, 553 aa, Artificial Sequence
  N267L_I291V
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLELHLFSIAVVVEPQYSQCRVAITKAVVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 36 - protein, 553 aa, Artificial Sequence
  N267S_I291T
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLESHLFSIAVVVEPQYSQCRVAITKATVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 37 - protein, 553 aa, Artificial Sequence
  N267S_I291T
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLETHLFSIAVVVEPQYSQCRVAITKATVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 38 - protein, 553 aa, Artificial Sequence
  N267S_I291S
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLESHLFSIAVVVEPQYSQCRVAITKASVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 39 - protein, 553 aa, Artificial Sequence
  N267S_I291C
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLESHLFSIAVVVEPQYSQCRVAITKACVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 40 - protein, 553 aa, Artificial Sequence
  N267L_I291S
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLELHLFSIAVVVEPQYSQCRVAITKASVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 41 - protein, 553 aa, Artificial Sequence
  Var1_291T
MDSVELRRTGIYHSTFWDIDSIRALLAKRDCTAAAAVSPEHHKKLKERIQKRLNDITQPHHLLGLIDAVQRLGVAYQFEEEISEALHGVHSENTDHAIKDSLHHTSLYFRLLRQHGCNLSSDIFNKFKKDGGGFRATLCEDAMGLLSLYEAVRLSVKGEAILEEAQVFSIANLKIVMERIERKLADRIEHALEIPLYWRAPRLEARWYIDVYEKDDGRIDDLLDFAKLDFNRVQMLYQTELKELSMWWELLGLPAKMGFFRDRLLENHLFSIAMLVEPQYSQCRVALTKATILISAMDDFYDIHGLPDELRVFTDTVNRWDLEGIEQLPDYMKVYYLALYNTTNETAWIILKEKGFNATHYLKKVWAMQSNAYFREAQWFNTGYIPKFDEYLENALVSVGAPFLLGVSYPLLQNQISKEELELLPEDLNLIRWASMIFRLWDDLATSKAEQQRGEVPKTIQCYMHETGSSEEVAANHIRDMISEAWKEVNAECLKPTSVSRHYVGVAPNTARSGLLMYHHDFDGFASPHGKTNAHITSIFFDPVPLKESINLG

SEQ ID NO: 42 - protein, 553 aa, Artificial Sequence
  Var2_291T
MDSMEVRKSAMYHSTFWDIDSIRAILAKRDCTAAAALSPEHHKRLKERIQKRLNDVTNPHHLLALIDAVQRLGVAYQFEEEISDALHGLHSEQSDHGLKDSLHHTSLYFRLLRQHGCNLSSDIFNKFKKEGGGFKATLCEDAMGLLSLYEAVRLSIRGEAILEEAQIFSIANLKIIIERLERKLAEKIEHALEIPLYWRAPRLEARWYIEIYEKDDGKIEDLLDFAKLDFNRMQMLYQTELKDLTMWWELLGLPAKMGFFRDRLLENHLFSIAMIVEPQYSNCRIGITKATVLVSAMDDFYDVHGLPDELRVFTETVNRWDLEGIDQLPDYMKVYYLGLYNTTNETAYLVVKDKGYNATHYLRKVWAMQSNAYFREAQWFNTGYIPKFDEYLDNAVVSVGAPFVVGLTYPLLQQQLTKEEIELIPEDLNLLRWASVIFRLYDDLATTKAEQQRGDMPKSIQCYMHETGTSEEVAANHIKEIISEAWKDVNAECLKPSSVSKHYLGLAPNSARSAMVIYHHDFDGFATPHGKTNAHITSIFFDPVPIKDSINLG

SEQ ID NO: 43 - protein, 553 aa, Artificial Sequence
  Var3_291T
MDSLDLRRSAIYHSTFWDIDSIKALIAKRDCSAAAAVSPEHHRKLKEKIQKKLQEISQPHHLLGLIDAVQRLGVAYQFEDEISDALHGMHTDQTDHAVKESLHHTSLWFRLLRQHGCNLSSDIFNRFRKEGGGFKASICEDAMGLLSLYEAVRLSIKGEAILEEAQVFSIANLRIMLEKVEKKLADRIDHALELPIFWRAPRLEARWYIEVYERDEGRIEELLDFAKLDFNRVQMVYQTELKELTMWWELVGLPARMGFFRDRILENHLFSLAILVEPQYSNCRVALTKATILISAMDDFYDVHGLPDELKLFTETVQRWDIDAIDNLPEYMKLFYVALYNTTNETAWLIVKERGFNATHYLRKVWAMQSNAYFREAQWFQSGYVPKFDEYLENAVISIGGPFILGLSYPLIQNQVTKEEIDLLPEDLNLVKWASVIFRLYDDLGTSKAENQRGDLPKSIQCYMHETGSSEEVAANHIKEMISEAWKDVNAECLRPSTISRHFLGVAPNSARSAVIMYHHDFDGFASPHAKTNAHITSIFYDPVPLRESINLG

SEQ ID NO: 44 - protein, 553 aa, Artificial Sequence
  Var1_291S
MDSMEIRRTAIYHSTFWDIDSIRAMLARRDCSAGAAVSPDHHKKLKEKIQRRLQDITQPHHLLGLIDAVQRLGVAYQFEDEISEALHGVHSENTEHAIKDSLHHTSLYFRLLRQHGCNLSSDIFNKFKKEGGGFKATLCEDAMGLLSLYEAVRLSIKGEAILEEAQVFSIANLRIMVEKLERKLADRIEHALEIPLYWRAPRLEARFYIEIYEKEDGKIDELLDFAKLDFNRVQMIYQTELKELTMWWELLGLPAKMGFFRDRLLENHLFSMAVIVEPQFSQCRLALTKASVLMTAMDDFYDVHGLPDELKVFTDTVNRWDLEGMDQLPEYMKLYYLALYNTTNETAYILVKEKGFNATHYLKRVWAMQSNGYYREAQWFNSGYLPKFDEYLDNALVSVGAPFLLGLSYPMMQQQITKEELDLIPEELNLVRWASIIFRLYDDLATSKAEQQRGDVPKSIQCYMHETGSSEEIAANHIKEMISEAWKDVNAECLKPTSVSKHFVGVAPNSARSGVVMYHHDFDGFASPHGRTNAHITSIFFEPVPIRDSINLG

SEQ ID NO: 45 - protein, 553 aa, Artificial Sequence
  Var2_291S
MDSVEVRRTGIYHSTFWDIDSIRALVARRDCSAGAALSPDHHKRLKERIQRRLQEISQPHHLLGLIDAVQRLGVAYQFEEEISDALHAIHTDQSDHAIRDSLHHTSLYFRLLRQHGCNLSSDIFQKFKKEGGGFKASLCEDAMGLLSLYEAVRLSIKGEAILEEAQVFSIANLRILLERIERKLAEKIEHALELPLYWRAPRLEARWYIEVYEKDDAKIEDLLDFAKLDFNRLQMMYQTELKELSMWWELLGLPARMGFFRDRILENHLFSIAMVVEPNYSQCRIALTKASILVSAMDDYYDVHGLPDELKIFTDTVNRWDLEGIEQLPDYMKIYYLALYNTTNETAYILLKEKGFNASHYLRKLWAVQSNAYFKEAQWFNSGYVPKFDEYLENALVSIGAPFIVAVTYPMIQNNVTKEEIELLPEDLQLLKWASVIFRLYDDLGTSKAEQQRGEVPKSIQCYMHETGSSEEVAANHIKEIISDAWKEVNAECLRPTTLSRHYVGVAPNSARSAVVIYHHDFDGFATPHAKTQAHITSIFFEPVPIKESINLG

SEQ ID NO: 46 - protein, 553 aa, Artificial Sequence
  Var3_291S
MDSLDLRRSAIYHSTFWDIDSIKALIAKRDCSAAAAVSPEHHRKLKEKIQKKLQEISQPHHLLGLIDAVQRLGVAYQFEDEISDALHGMHTDQTDHAVKESLHHTSLWFRLLRQHGCNLSSDIFNRFRKEGGGFKASICEDAMGLLSLYEAVRLSIKGEAILEEAQVFSIANLRIMLEKVEKKLADRIDHALELPIFWRAPRLEARWYIEVYERDEGRIEELLDFAKLDFNRVQMVYQTELKELTMWWELVGLPARMGFFRDRILENHLFSLAILVEPQYSNCRVALTKASILISAMDDFYDVHGLPDELKLFTETVQRWDIDAIDNLPEYMKLFYVALYNTTNETAWLIVKERGFNATHYLRKVWAMQSNAYFREAQWFQSGYVPKFDEYLENAVISIGGPFILGLSYPLIQNQVTKEEIDLLPEDLNLVKWASVIFRLYDDLGTSKAENQRGDLPKSIQCYMHETGSSEEVAANHIKEMISEAWKDVNAECLRPSTISRHFLGVAPNSARSAVIMYHHDFDGFASPHAKTNAHITSIFYDPVPLRESINLG

SEQ ID NO: 47 - protein, 553 aa, Artificial Sequence
  Var1_291C
MDSMEVRRTAIYHSTFWDIDSIRALIARRDCTAAAAISPDHHKRLKERIQKRLQDITQPHHLLGLIDAVQRLGVAYQFEEEISDALHGLHSENTEHGIRDSLHHTSLYFRLLRQHGCNLSSDIFNKFKKDGGGFKATLCDDAMGLLSLYEAVRLSVKGEAILEEAQVFSIANLKILLDRLDRKLAERIEHALDLPLFWRAPRLEARWYIDLYEKDDGRIDDLLDFAKLDFNRVQLIYQTELKELSMWWELVGLPAKMGFFRDRILENHLFTVAIVVEPQYSQCRLAITKACILMTAMDDFYDVHGLPDELKLFTDTVQRWDIEAMEQLPEYMKIYYLALYNTTNETAYVILKEKGFNATHYLKKVWAMQSNAYFREAQWFNSGYVPKFDEYLDNALVSVGAPFVLGVTYPVIQQQLSKEELDLVPEDLNLVKWASIIFRLYDDLATSKAEQQRGDVPKSIQCYMHETGSSEEVAANHIRDMISDAWKDVNAECLKPTSLSKHYLGLAPNSARSGVLLYHHDFDGFATPHARTNGHITSIFFDPVPLKESINLG

SEQ ID NO: 48 - protein, 553 aa, Artificial Sequence
  Var2_291C
MDSVEVRRTGIYHSTFWDIDSIRALVARRDCSAGAALSPDHHKRLKERIQRRLQEISQPHHLLGLIDAVQRLGVAYQFEEEISDALHAIHTDQSDHAIRDSLHHTSLYFRLLRQHGCNLSSDIFQKFKKEGGGFKASLCEDAMGLLSLYEAVRLSIKGEAILEEAQVFSIANLRILLERIERKLAEKIEHALELPLYWRAPRLEARWYIEVYEKDDAKIEDLLDFAKLDFNRLQMMYQTELKELSMWWELLGLPARMGFFRDRILENHLFSIAMVVEPNYSQCRIALTKACILVSAMDDYYDVHGLPDELKIFTDTVNRWDLEGIEQLPDYMKIYYLALYNTTNETAYILLKEKGFNASHYLRKLWAVQSNAYFKEAQWFNSGYVPKFDEYLENALVSIGAPFIVAVTYPMIQNNVTKEEIELLPEDLQLLKWASVIFRLYDDLGTSKAEQQRGEVPKSIQCYMHETGSSEEVAANHIKEIISDAWKEVNAECLRPTTLSRHYVGVAPNSARSAVVIYHHDFDGFATPHAKTQAHITSIFFEPVPIKESINLG

SEQ ID NO: 49 - protein, 553 aa, Artificial Sequence
  Var3_291C
MDSIDIRRTAIYHSTFWDIDSIRALLAKRECSAAGAMSPDHHKKLKEKIQKKLQELTQPHHLLALIDAVQRLGVAYQFEDEISEALHGIHSDNSEHAIRESLHHTSLYFRLLRQHGCNLSTDIFNKFRKDGGGFKATLCEDAMGLLSLYEAVRLSVRGEAILEEAQIFSIANLRIVIEKIERKLADKIEHALELPLYWRAPRLEARWYIDIYEKDEGRIEDLLDFAKLDFNRVQLIYQTELRELTMWWELLGLPAKMGFFRDRLLENHLFSVAMLVEPNYSQCRVALTKACVLVSAMDDFYDIHGLPDELRLFTETVQRWDLDALDQLPEYMKIFYMALYNTTNETAYLMLKDKGFNASHYLKKVWALQSNGYFREAQWYNSGYVPKFDEYLDNAVISVGGPFLLGVSYPLLQNQLTKEEMELLPEDLNLVKFASVIFRLYDDLATSKAEQNRGDLPKSIQCYMHETGSSEEMAAQHIKDIISEGWKDVNAECLRPSSVSRHFLGLAPNSARSGVIVYHHDFDGFASPHARSNAHITSIFFEPVPIRESINLG

SEQ ID NO: 50 - protein, 553 aa, Artificial Sequence
  Var_267S_291T
MDSIDIRRTAIYHSTFWDIDSIRALLAKRECSAAGAMSPDHHKKLKEKIQKKLQELTQPHHLLALIDAVQRLGVAYQFEDEISEALHGIHSDNSEHAIRESLHHTSLYFRLLRQHGCNLSTDIFNKFRKDGGGFKATLCEDAMGLLSLYEAVRLSVRGEAILEEAQIFSIANLRIVIEKIERKLADKIEHALELPLYWRAPRLEARWYIDIYEKDEGRIEDLLDFAKLDFNRVQLIYQTELRELTMWWELLGLPAKMGFFRDRLLESHLFSVAMLVEPNYSQCRVALTKATVLVSAMDDFYDIHGLPDELRLFTETVQRWDLDALDQLPEYMKIFYMALYNTTNETAYLMLKDKGFNASHYLKKVWALQSNGYFREAQWYNSGYVPKFDEYLDNAVISVGGPFLLGVSYPLLQNQLTKEEMELLPEDLNLVKFASVIFRLYDDLATSKAEQNRGDLPKSIQCYMHETGSSEEMAAQHIKDIISEGWKDVNAECLRPSSVSRHFLGLAPNSARSGVIVYHHDFDGFASPHARSNAHITSIFFEPVPIRESINLG

SEQ ID NO: 51 - protein, 553 aa, Artificial Sequence
  Var_267S_291S
MDSIDIRRTAIYHSTFWDIDSIRALLAKRECSAAGAMSPDHHKKLKEKIQKKLQELTQPHHLLALIDAVQRLGVAYQFEDEISEALHGIHSDNSEHAIRESLHHTSLYFRLLRQHGCNLSTDIFNKFRKDGGGFKATLCEDAMGLLSLYEAVRLSVRGEAILEEAQIFSIANLRIVIEKIERKLADKIEHALELPLYWRAPRLEARWYIDIYEKDEGRIEDLLDFAKLDFNRVQLIYQTELRELTMWWELLGLPAKMGFFRDRLLESHLFSVAMLVEPNYSQCRVALTKASVLVSAMDDFYDIHGLPDELRLFTETVQRWDLDALDQLPEYMKIFYMALYNTTNETAYLMLKDKGFNASHYLKKVWALQSNGYFREAQWYNSGYVPKFDEYLDNAVISVGGPFLLGVSYPLLQNQLTKEEMELLPEDLNLVKFASVIFRLYDDLATSKAEQNRGDLPKSIQCYMHETGSSEEMAAQHIKDIISEGWKDVNAECLRPSSVSRHFLGLAPNSARSGVIVYHHDFDGFASPHARSNAHITSIFFEPVPIRESINLG

SEQ ID NO: 52 - protein, 553 aa, Artificial Sequence
  Var_267L_291S
MDSLDLRRSAIYHSTFWDIDSIKALIAKRDCSAAAAVSPEHHRKLKEKIQKKLQEISQPHHLLGLIDAVQRLGVAYQFEDEISDALHGMHTDQTDHAVKESLHHTSLWFRLLRQHGCNLSSDIFNRFRKEGGGFKASICEDAMGLLSLYEAVRLSIKGEAILEEAQVFSIANLRIMLEKVEKKLADRIDHALELPIFWRAPRLEARWYIEVYERDEGRIEELLDFAKLDFNRVQMVYQTELKELTMWWELVGLPARMGFFRDRILELHLFSLAILVEPQYSNCRVALTKASILISAMDDFYDVHGLPDELKLFTETVQRWDIDAIDNLPEYMKLFYVALYNTTNETAWLIVKERGFNATHYLRKVWAMQSNAYFREAQWFQSGYVPKFDEYLENAVISIGGPFILGLSYPLIQNQVTKEEIDLLPEDLNLVKWASVIFRLYDDLGTSKAENQRGDLPKSIQCYMHETGSSEEVAANHIKEMISEAWKDVNAECLRPSTISRHFLGVAPNSARSAVIMYHHDFDGFASPHAKTNAHITSIFYDPVPLRESINLG

SEQ ID NO: 53 - protein, 553 aa, Artificial Sequence
  CiCassy I291L
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLENHLFSIAVVVEPQYSQCRVAITKALVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 54 - protein, 11 aa, Artificial Sequence
  RRX8W motif
  VARIANT 3: any amino acid
  VARIANT 4: any amino acid
  VARIANT 5: any amino acid
  VARIANT 6: any amino acid
  VARIANT 7: any amino acid
  VARIANT 8: any amino acid
  VARIANT 9: any amino acid
  VARIANT 10: any amino acid
RRXXXXXXXXR

SEQ ID NO: 55 - protein, 11 aa, Artificial Sequence
  R(R/K)X8W motif
  VARIANT 2: Xaa is Arginine or Lysine
  VARIANT 3: any amino acid
  VARIANT 4: any amino acid
  VARIANT 5: any amino acid
  VARIANT 6: any amino acid
  VARIANT 7: any amino acid
  VARIANT 8: any amino acid
  VARIANT 9: any amino acid
  VARIANT 10: any amino acid
RXXXXXXXXXR

SEQ ID NO: 56 - protein, 553 aa, Artificial Sequence
  N267W
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLEWHLFSIAVVVEPQYSQCRVAITKAIVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 57 - protein, 553 aa, Artificial Sequence
  N267G
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLEGHLFSIAVVVEPQYSQCRVAITKAIVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 58 - protein, 553 aa, Artificial Sequence
  N267A
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLEAHLFSIAVVVEPQYSQCRVAITKAIVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG